(19) United States
(12) Patent Application Publication
(10) Pub. No.: US 2022/0118002 A1
(43) Pub. Date: Apr. 21, 2022

(54) COMPOSITIONS OF MATTER WITH ACTIVITY TO REMOVE LIPOFUSCIN FROM RETINAL CELLS

(71) Applicant: Cornell University, Ithaca, NY (US)

(72) Inventors: Marcelo M. Nociari, Ithaca, NY (US); Enrique Rodriguez Boulan, Ithaca, NY (US)

(73) Assignee: Cornell University, Ithaca, NY (US)

(21) Appl. No.: 17/436,023

(22) PCT Filed: Mar. 3, 2020
(86) PCT No.: PCT/US2020/020805

§ 371 Date: Sep. 2, 2021

Related U.S. Application Data
(60) Provisional application No. 62/814,028, filed on Mar. 5, 2019.

(51) Int. Cl. A61K 31/724; A61P 27/02; A61K 47/40; A61K 47/26; A61K 9/00

(52) U.S. Cl. CPC A61K 31/724; A61P 27/02; A61K 9/0048; A61K 47/26; A61K 47/40

(57) ABSTRACT

The present disclosure relates generally to compositions and methods for the treatment of eye diseases (e.g., retinopathies), and more particularly, to treatment of eye diseases associated with retinal cell lipofuscin accumulation.

### CROSS-REFERENCE TO RELATED APPLICATIONS

This application claims the benefit of and priority to U.S. Provisional Patent Application No. 62/814,028, filed Mar. 5, 2019, the entire contents of which are incorporated herein by reference.

### GOVERNMENT SUPPORT

This invention was made with government support under grant number EY027422-02, awarded by the National Eye Institute (NEI)/National Institutes of Health (NIH). The government has certain rights in the invention.

### TECHNICAL FIELD

The present disclosure relates generally to compositions and methods for treating eye diseases (e.g., retinopathies), and more particularly, eye diseases associated with retinal cell lipofuscin accumulation.

### BACKGROUND

The following description of the background of the present technology is provided simply as an aid in understanding the present technology and is not admitted to describe or constitute prior art to the present technology.

Lipofuscin is a fine yellow-brown pigment composed of indigestible material that is believed to be remnants after lysosomal digestion. Lipofuscin is mostly composed of dimers of retinaldehydes known as lipid bisretinoids, and small amounts of carbohydrates, oxidized proteins and metals. Accumulation of lipofuscin in retinal cells causes retinal toxicity, which is associated with conditions like macular degeneration, a degenerative disease of the eye, and Stargardt disease.

### SUMMARY OF THE PRESENT TECHNOLOGY

In one aspect, the present technology provides a method for preventing or treating an eye disease associated with retinal cell lipofuscin accumulation without impairing visual acuity in a subject in need thereof comprising administering to the subject an effective amount of sulfobutyl ether β-Cyclodextrin (SBE-βCD) or a pharmaceutically acceptable salt thereof. The eye disease associated with retinal cell lipofuscin accumulation may be selected from the group consisting of Stargardt disease (STGD), retinitis pigmentosa (RP), Age-Related Macular Degeneration (AMD), Best disease (BD), and cone-rod dystrophy. Additionally or alternatively, in some embodiments, the eye disease is genetic, non-genetic, or associated with aging. In another aspect, the present technology provides a method for preventing or treating retinal cell lipofuscin accumulation without impairing visual acuity in a subject in need thereof comprising administering to the subject an effective amount of sulfobutyl ether β-Cyclodextrin (SBE-βCD) or a pharmaceutically acceptable salt thereof.

Additionally or alternatively, in some embodiments of the methods disclosed herein, administration of the effective amount of the SBE-βCD or pharmaceutically acceptable salt thereof prevents exacerbation of lipofuscin-associated retinal damage in the subject.

Also disclosed herein are methods for reducing lipofuscin accumulation in retinal pigment epithelium cells comprising contacting the retinal pigment epithelium cells with an effective amount of sulfobutyl ether β-Cyclodextrin (SBE-βCD) or a pharmaceutically acceptable salt thereof.

In any and all embodiments of the methods disclosed herein, the SBE-βCD or pharmaceutically acceptable salt thereof is configured to localize to retinal pigment epithelium cells. In certain embodiments, the SBE-βCD or pharmaceutically acceptable salt thereof is configured to complex with lipofuscin bisretinoid lipids in the retinal pigment epithelium cells. The lipofuscin bisretinoid lipids may be N-retinylidene-N-retinylethanolamine (A2E), an A2E isomer, an oxidized derivative of A2E, or all-trans-retinal dimers.

Additionally or alternatively, in some embodiments of the methods disclosed herein, administration of the effective amount of the SBE-βCD or pharmaceutically acceptable salt thereof blocks, mitigates, or reverses accumulation of lipofuscin in retinal pigment epithelium cells.

In any of the preceding embodiments of the methods disclosed herein, the SBE-βCD or pharmaceutically acceptable salt thereof is coupled to an agent that targets retinal pigment epithelium cells. In some embodiments, the agent targets endosomes or lysosomes in the retinal pigment epithelium cells, such as mannose 6-phosphate. Additionally or alternatively, in some embodiments of the methods disclosed herein, the SBE-βCD or pharmaceutically acceptable salt thereof is coupled to a fluorophore. Examples of fluorophores include, but are not limited to fluorescein, rhodamine, Oregon green, eosin, Texas Red, cyanine, streptocyanines, hemicyanines, closed chain cyanines, phycocyanins, allophycocyanins, indocarbocyanines, oxacarbocyanines, thiacarbocyanines, merocyanins, and phthalocyanines, naphthalene derivatives (e.g., dansyl and prodan derivatives), coumarin and its derivatives, oxadiazole and its derivatives (e.g., pyridyloxazoles, nitrobenzoxadiazoles, and benzoxadiazoles), pyrene and its derivatives, oxazine and its derivatives (e.g., Nile Red, Nile Blue, and cresyl violet), acridine derivatives (e.g., proflavin, acridine orange, and acridine yellow), arylmethine derivatives (e.g., auramine, crystal violet, and malachite green), and tetrapyrrole derivatives (e.g., porphyrins and bilirubins).

Additionally or alternatively, in some embodiments of the methods disclosed herein, the SBE-βCD or pharmaceutically acceptable salt thereof is administered via topical, intravitreous, intraocular, subretinal, or subscleral administration. In certain embodiments, subscleral administration is achieved by implanting a slow-release subscleral implant in the subject.

### BRIEF DESCRIPTION OF THE DRAWINGS

FIGS. 1A-1B show the structural formulae of the compositions of the present technology. FIG. 1A shows the general formula for β (beta)-Cyclodextrins, which contain seven substituted glucopyranoside units. Substitutions are indicated by the symbol R. FIG. 1B shows the identities of the substituents in (i) Methyl beta-Cyclodextrin (MβCD or MBCD); (ii) 2-hydroxypropyl beta-Cyclodextrin (HPβCD); and (iii) Sulfobutyl ether β-Cyclodextrin (SBEβCD).

FIG. 2 shows the removal of LB using the different cyclodextrins of the present technology at a dose of 4 mM. Human RPE cultured cells were pre-loaded with lipofuscin by incubating them with 5 μM A2E overnight. Cells were washed and left untreated or treated with 4 mM of a cyclodextrin compound (indicated on the X-axis). At 24 hrs., cells were harvested with trypsin, pelleted down and lysed in 2% Triton-buffer. A2E content was assessed by fluorescence at 430 nm and cell number was obtained by interpolating the protein content into a calibration curve of protein content vs cell-number. 100% corresponds to the content in unextracted cells. The columns are labeled as follows: A0=α-Cyclodextrin; B0=β-Cyclodextrin; M1=Methyl β-Cyclodextrin; M2=Methyl β-Cyclodextrin; M3=Methyl β-Cyclodextrin; M4=Heptakis (2,6-di-O-Methyl) β-Cyclodextrin; M5=Heptakis (2,3,6-tri-O-Methyl) β-Cyclodextrin; H1=Hydroxy propyl β-Cyclodextrin; H2=Hydroxy propyl β-Cyclodextrin; H3=Hydroxy propyl β-Cyclodextrin; SB1=Sulfo Butyl Ether β-Cyclodextrin; SB2=Sulfo Butyl Ether β-Cyclodextrin; SB3=Sulfo Butyl Ether β-Cyclodextrin; G0=Gamma-cyclodextrin. As shown in FIG. 2, not all cyclodextrins are effective in promoting LB removal. Whereas methyl β-Cyclodextrin, 2-Hydroxy Propyl β-Cyclodextrin, and Sulfo Butyl Ether β-Cyclodextrin promote removal of lipofuscin bisretinoids from retinal cells, unsubstituted α-Cyclodextrin, β-Cyclodextrin and gamma-cyclodextrin did not extract A2E under these conditions. 2,6-di-O-methyl-β-Cyclodextrin (Heptakis 2,6-di-O-methyl) or 2,3,6-tri-O-methyl-β-Cyclodextrin (Heptakis 2,3,6-tri-O-methyl) were also not effective in promoting LB removal. *100 mM Stock was prepared in DMSO, due to lack of water solubility.

FIG. 3A shows the dose-dependent removal of LB with methyl β-Cyclodextrin in the millimolar range. Epithelial cells loaded with A2E were treated with the indicated doses of methyl β-Cyclodextrin for 24 hours. As a control, untreated epithelial cells, which were not preloaded with A2E, were treated with the indicated doses of methyl β-Cyclodextrin for 24 hours. A2E content per cell at the end of the incubation period was plotted as a function of the dose of methyl β-Cyclodextrin.

FIG. 3B shows removal of LB with methyl β-Cyclodextrin. Epithelial cells loaded with A2E were treated with methyl β-Cyclodextrin for 24 hours. At the end of the incubation period, fluorescence of A2E inside the cells was monitored by fluorescence microscopy at 100× magnification. Cells were stained with Hoechst stain to visualize nuclei of cells that contained A2E. Left column shows lipofuscin LB accumulation in an untreated control (A2E-Untreated), as shown by fluorescence for LB (turquoise, arrows) and nucleus (blue, arrow heads). Right column shows LB accumulation (turquoise, arrows) and nucleus (blue, arrow heads) after the treatment with methyl β-Cyclodextrin.

FIGS. 4A-4B show the removal of LB with methyl β-Cyclodextrin as determined by fluorescence microscopy. Epithelial cells loaded with A2E were treated with methyl β-Cyclodextrin for 24 hours. Cells were stained with Hoechst stain to visualize nuclei. Fluorescence of A2E inside the cells (unextracted cells) was monitored by fluorescence microscopy at 100× magnification. FIG. 4A shows LB-rich lipofuscin accumulation in an untreated control (A2E-Untreated), as shown by fluorescence for LB and nucleus. FIG. 4B shows LB expelled from cells after treatment with 4 mM MβCD for 24 hrs.

FIG. 5A shows the assays used to identify agents that remove lipofuscin in retinal cells. Human retinal pigment epithelium (RPE) cultured cells, ARPE-19 (ATCC), were pre-loaded with lipofuscin by incubating them with 5 μM A2E (the most abundant LB in retinal RPE), overnight. A2E was used as a surrogate of the LB-rich lipofuscin found in the RPE of the eye. Cells were plated into 96 well plates at 1×10⁵ cells per well. After 48 hrs in DMEM-10% media, the media was replaced with DMEM-10% media either with or without a test agent in a 24 hr removal assay. Each dose of removal agent was evaluated in quadruplicate. At the end of the treatment, the supernatants were removed and adherent cells were lysed with 1×A2E-solubilizing buffer. A2E content was determined based on the fluorescence of the lysates using a spectrophotometer plate reader (excitation: 430 nm, emission 600 nm).

FIG. 5B plots the correlation between picomoles of A2E per cell and fluorescence of known amounts of an A2E standard. To calculate picomoles of A2E per cell, A2E-specific fluorescence values (fluorescence of lysates from cells pre-loaded with A2E minus fluorescence of corresponding lysates from control cells with no A2E loaded) were interpolated into calibration curves generated by plotting fluorescence (430 nm ex./600 nm em) versus known amounts of an A2E standard dissolved in 1×A2E-solubilizing buffers. The composition of the A2E-solubilizing buffer was optimized to eliminate solvatochromic interferences with cyclodextrins that could disturb the calculations of the amount of A2E, using fluorescence.

FIG. 5C shows that under the conditions described in FIG. 5B, the fluorescence of A2E is not affected by cyclodextrin complexation and that the fluorescence readout reflects the true A2E amounts.

FIG. 6 shows images of LB removal with methyl β-Cyclodextrin, 2-Hydroxy Propyl β-Cyclodextrin, and Sulfo Butyl Ether β-Cyclodextrin. Untreated A2E-loaded RPE cultures served as negative controls. A2E-loaded RPE cultures were treated with 7.5 mM of the indicated β-Cyclodextrin compound for 48 hrs. For each cyclodextrin, three separate lots were tested. Lipofuscin (yellow) is visualized by fluorescence microscopy (488 nm/600 nm) using low magnification (10×) objective to capture a large number of cells.

FIG. 7 shows examples of the stoichiometric distribution of A2E at the end of the treatment with β-Cyclodextrins. Epithelial cells pre-loaded with A2E were treated in serum free media with 12.5 mM of the indicated β-Cyclodextrins for 4 hrs. A2E content in cell lysates and supernatants were determined by fluorescence assay as described in FIG. 5A. As shown in FIG. 7, the majority of A2E disappearing from the cells appeared in the supernatant.

FIG. 8 shows that β-Cyclodextrins exert a dose-dependent LB removal effect. Epithelial cells loaded with A2E were treated with the indicated doses of β-Cyclodextrins for 48 hours. As a control, untreated RPE cells, which were not preloaded with A2E, were treated with the indicated doses of β-Cyclodextrins for 48 hours. At the end of the incubation, cells were lysed with A2E-solubilizing buffer and the A2E-specific fluorescence (ASF) for each drug concentration was calculated by subtracting the fluorescence of the control lysates from the fluorescence in the A2E-loaded cells lysate treated with the same cyclodextrin dose. ASF was interpolated in an A2E standard curve to convert to picomoles. Percentage of A2E-content per cell was plotted as a function of the dose of β-Cyclodextrin.

FIG. 9 shows the quantification of in vivo removal by methyl β-Cyclodextrin (M-βCD), 2-Hydroxy Propyl β-Cyclodextrin (HP-βCD), and Sulfo Butyl Ether β-Cyclodextrin (SEB-βCD). Animals with aberrant LB accumulation, due to a double mutation (DKO) in the pathway that recycles retinaldehydes in the RPE, received two intravitreal injections per eye, one week apart from each other, of either: 2 μl 100 mM M-βCD (right eye, OD); 1 μl 500 mM HP-βCD or 1 μl 500 mM SEB-βCD, in water. A separated age-matched group was injected with the corresponding volume of vehicle (water), as control. Eyes were harvested 4 days after the second injection. Retinal pigment epithelium (RPE)-eyecups were flat mounted and subjected to autofluorescence microscopy at 63× magnification. Multiple pictures were stitched together to show the complete eyecups. Stitched images were converted to gray scale and mean fluorescence intensity of the retinas were measured using Image J (NIH). Due to solubility constraints, M-PCD stocks were prepared at 100 mM and 2 μl were administered for in vivo treatments. Lipofuscin (%) remaining in the eyes were compared to eyes from animals that received the same volume of vehicle (water) in their eyes. Significance was determined by 2 tails, unpaired T-tests. While M-βCD did not significantly reduce the amount of lipofuscin, HP-βCD and SEB-βCD were effective.

FIGS. 10A-10B show that the removal of LB with SEB-βCD did not impair visual acuity. To measure the effects of β-Cyclodextrin treatment on visual function, spatial frequency (SF) tests (a measure of visual acuity) were performed. SF was assessed by OptoMotry (Prusky G T, et al. (2004) Rapid quantification of adult and developing mouse spatial vision using a virtual optomotor system. Invest Ophthalmol Vis Sci 45(12):4611-6; Douglas R M, et al. (2005) Independent visual threshold measurements in the two eyes of freely moving rats and mice using a virtual-reality optokinetic system. Vis Neurosci 22(5):677-84; Kretschmer F, et al. (2015) A system to measure the Optokinetic and Optomotor response in mice. J Neurosci Methods 256:91-105). Briefly, the unrestrained mouse stands on an elevated platform surrounded by computer monitors that create a virtual cylinder with black bars on white background. The virtual rotation of the grating triggers visual reflexive head movements; the frequency of the black bars can be changed by adjusting the thickness of the bars. Thinner bars, i.e. higher SF, are equivalent to the smaller letters in the Snellen chart used to assess visual acuity in humans. The highest SF that does not elicit tracking is taken as a measure of visual acuity. Because Methyl β-Cyclodextrin treatment did not significantly reduce the amount of lipofuscin at the tested dose, SF was not evaluated. Hydroxy propyl β-Cyclodextrin which was moderately effective removing LBs resulted in significant impairment of visual acuity at the dose used during the experiment, while Sulfo Butyl Ether β-Cyclodextrin was well tolerated as it did not affect the vision of the mice.

FIG. 11A shows that treatment with Sulfo Butyl Ether β-Cyclodextrin (SBE-BCD) does not compromise the integrity of the photoreceptor layer (ONL). Morphometric analysis comparing the thickneness of the outer nuclear layer (ONL) in control and SBE-BCD treated animals was performed. The layer of photoreceoptors was selected using Photoshop and ImageJ and thickness was measured.

FIG. 11B shows that the retinas of control and Sulfo Butyl Ether β-Cyclodextrin (SBE-BCD) treated animals were intact following treatment. Stitched images were created using ZEN Blue (Zeiss) from individual fields obtained by color microscopy of hematoxylin and eosin stained paraffin-embedded cross sections of retinas from mice treated with 1 μl vehicle or Sulfo Butyl β-Cyclodextrin.

### DETAILED DESCRIPTION

It is to be appreciated that certain aspects, modes, embodiments, variations and features of the present methods are described below in various levels of detail in order to provide a substantial understanding of the present technology.

Toxic lipid bisretinoids (LB), the main components of eye lipofuscin, accumulate with age in the lysosomes of Retinal Pigment Epithelium, a major retinal support cell responsible for the survival of photoreceptors, the light sensing cells. LB accumulation in RPE is enhanced in human genetic diseases such as Stargardt disease (STGD), ABCA4-related forms of cone-rod dystrophy (CRD), and retinitis pigmentosa (RP). Such accumulation is believed to be toxic for RPE and to be a key aspect of the etiology of these diseases. Furthermore, accumulation of LB with age is a risk factor for Age-Related Macular Degeneration (AMD), an incurable disease that affects 25% of the senior population. Currently, there are no medical treatments to revert LB accumulation. Therefore, agents that remove cellular LB have potential to fulfill an unmet need in the treatment of human blinding diseases.

Cyclodextrins are cyclic oligosaccharides containing glucose subunits joined by α-1,4 glycosidic bonds. Cyclodextrins are produced from starch by enzymatic conversion, and are classified as α-cyclodextrin, as containing six glucose subunits, β-Cyclodextrin, as containing seven glucose subunits, and γ-cyclodextrin, as containing 8 glucose subunits. α-, β-, as well as γ-cyclodextrins are all generally recognized as safe by the Food and Drug Administration (FDA), and are commonly used in food, pharmaceutical, drug delivery, and chemical industries, as well as agriculture and environmental engineering. Among other derivatives, methyl beta-Cyclodextrin (M-βCD) and 2-hydroxypropyl beta-Cyclodextrin (HP-βCD) are considered safe. HP-βCD has received IND status from FDA for the treatment of Niemann-Pick Type C1 Disease.

Previous studies discovered that certain cyclodextrins were capable of encapsulating and removing LB from cells. See Davis M E, Brewster M E, Cyclodextrin-based pharmaceutics: past, present and future. Nat Rev Drug Discov 3(12):1023-35 (2004); Nociari et al., Beta cyclodextrins bind, stabilize, and remove lipofuscin bisretinoids from retinal pigment epithelium. Proc Natl Acad Sci USA. 111(14): E1402-E1408 (2014).

The present disclosure is based, in part, on the discovery that not all cyclodextrins are effective in promoting in vivo removal of lipofuscin from epithelial cells. It was unexpectedly discovered that (i) Methyl beta-Cyclodextrin (MβCD); (ii) 2-hydroxypropyl beta-Cyclodextrin (HPβCD); and (iii) Sulfobutyl ether β-Cyclodextrin (SBEβCD) are effective in promoting LB removal, whereas other derivatives such as alpha-cyclodextrin, gamma-cyclodextrin, 2,6-di-O-methyl-β-Cyclodextrin (Heptakis 2,6-di-O-methyl) and 2,3,6-tri-O-methyl-β-Cyclodextrin (Heptakis 2,3,6-tri-O-methyl) fail to effectively remove cellular LB. Moreover, both methyl β-Cyclodextrin and hydroxy propyl β-Cyclodextrin produced significant impairment of visual acuity in vivo, while Sulfo Butyl Ether β-Cyclodextrin did not impair visual acuity.

Accordingly, the present disclosure provides methods for treating a subject suffering from an eye disease associated with retinal cell lipofuscin accumulation. Conditions and diseases treatable by the method described herein include any ophthalmologic or retinal disorder, condition, or disease directly or indirectly caused by the accumulation of lipofuscin in retinal pigment epithelium (RPE) cells, which may be genetic or non-genetic, such as age-related macular degeneration (AMD), Stargardt disease (SD), Best disease (BD), retinitis pigmentosa, and cone-rod dystrophy.

### Definitions

Unless defined otherwise, all technical and scientific terms used herein generally have the same meaning as commonly understood by one of ordinary skill in the art to which this technology belongs. As used in this specification and the appended claims, the singular forms “a” “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, or topically. Administration includes self-administration and the administration by another.

As used herein, the term “biological sample” means sample material derived from living cells. Biological samples may include tissues, cells, protein or membrane extracts of cells, and biological fluids (e.g., ascites fluid or cerebrospinal fluid (CSF)) isolated from a subject, as well as tissues, cells and fluids present within a subject. Biological samples of the present technology include, but are not limited to, samples taken from eye, breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, thymus, blood, hair, buccal, skin, serum, plasma, CSF, semen, prostate fluid, seminal fluid, urine, feces, sweat, saliva, sputum, mucus, bone marrow, lymph, and tears. Biological samples can also be obtained from biopsies of internal organs. Biological samples can be obtained from subjects for diagnosis or research or can be obtained from non-diseased individuals, as controls or for basic research. Samples may be obtained by standard methods including, e.g., venous puncture and surgical biopsy. In certain embodiments, the biological sample is a tissue sample obtained by needle biopsy.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, the terms “individual”, “patient”, or “subject” can be an individual organism, a vertebrate, a mammal, or a human. In some embodiments, the individual, patient or subject is a human.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration. Pharmaceutically-acceptable carriers and their formulations are known to one skilled in the art and are described, for example, in Remington's Pharmaceutical Sciences (20th edition, ed. A. Gennaro, 2000, Lippincott, Williams & Wilkins, Philadelphia, Pa.). Examples of pharmaceutically-acceptable carriers include a liquid or solid filler, diluent, excipient, manufacturing aid (e.g., lubricant, talc magnesium, calcium or zinc stearate, or steric acid), or solvent encapsulating material, useful for introducing the active agent into the body.

As used herein, “prevention,” “prevent,” or “preventing” of a disorder or condition refers to one or more compounds that, in a statistical sample, reduces the occurrence of the disorder or condition in the treated sample relative to an untreated control sample, or delays the onset of one or more symptoms of the disorder or condition relative to the untreated control sample.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

Eye Diseases Associated with Retinal Cell Lipofuscin Accumulation

Macular conditions that are associated with excessive accumulation of lipofuscin in retinal pigment epithelial (RPE) cells of the eye lead to blindness. Macular conditions that exhibit lipofuscin accumulation include Best macular dystrophy, Stargardt disease/Fundus flavimaculatus, ABCA4-related retinitis pigmentosa, ABCA4-related rod-cone dystrophy, and age-related macular degeneration (AMD). Lipofuscin is resistant to lysosomal enzyme degradation, and is believed to be the remnant of lysosomal digestion. Lipofuscin in the eye contains mostly lipid bisretinoids A2E, isoA2E, and all-trans-retinal dimer-phosphatidylethanolamine. Lipofuscin also contains small amounts of peptides featuring oxidative modifications, like nitrotyrosine, generated from reactive nitrogen oxide species and carboxyethylpyrrole, and iso[4]levuglandin E2 adducts generated from reactive lipid fragments, etc. See, e.g., Ng K-P, et al. (2008) Retinal Pigment Epithelium Lipofuscin Proteomics. Mol Cell Proteomics 7(7):1397-1405; Ben-Shabat S, et al. Fluorescent pigments of the retinal pigment epithelium and age-related macular degeneration. Bioorg Med Chem Lett 11(12):1533-40 (2001).

Lipofuscin accumulates with age and can increase due to genetic predispositions and certain underlying conditions. See Molday R S, Zhong M, Quazi F, The role of the photoreceptor ABC transporter ABCA4 in lipid transport and Stargardt macular degeneration. Biochim Biophys Acta 1791(7):573-83 (2009); Zaneveld J, et al. Comprehensive analysis of patients with Stargardt macular dystrophy reveals new genotype-phenotype correlations and unexpected diagnostic revisions. Genet Med 17(4):262-270 (2015); Allikmets R et al., Mutation of the Stargardt disease gene (ABCR) in age-related macular degeneration, Science 277(5333):1805-7 (1997); van Driel M a, Maugeri a, Klevering B J, Hoyng C B, Cremers F P, ABCR unites what ophthalmologists divide(s). Ophthalmic Genet 19(3):117-122 (1998); Fishman G A, Historical evolution in the understanding of Stargardt macular dystrophy. Ophthalmic Genet 31(4):183-9 (2010); Lim L S, Mitchell P, Seddon J M, Holz F G, Wong T Y, Age-related macular degeneration. Lancet 379(9827):1728-1738 (2012); Swaroop A, Chew E Y, Rickman C B, Abecasis G R, Unraveling a multifactorial late-onset disease: from genetic susceptibility to disease mechanisms for age-related macular degeneration. Annu Rev Genomics Hum Genet 10:19-43 (2009); Charbel Issa P, Barnard A R, Herrmann P, Washington I, MacLaren RE (2015) Rescue of the Stargardt phenotype in Abca4 knockout mice through inhibition of vitamin A dimerization. Proc Natl Acad Sci 112(27):8415-20 (2017).

### Current Therapeutic Strategies for Targeting Retinal Cell Lipofuscin Accumulation

Pharmacological therapies targeting LB deposits that are currently under development include strategies that block de novo formation of LB, and strategies for removing LB. The strategies that block de novo formation of LB include administration of fenretinide, emixustat hydrochloride, deuterated vitamin A, and aldehyde traps. The disadvantage of such strategies is that although they may prevent de novo formation of LBs, they cannot reduce pre-existing LB deposits. Therefore, strategies that block de novo formation of LB would provide little or no benefit for patients with established clinical symptoms. Strategies for removing LB include administration of soraprazan, and enzymatic degradation of LB. None of these existing strategies have progressed enough to confer a clinical benefit on patients.

Among the strategies that block de novo formation of LB, oral administration of fenretinide, a synthetic form of vitamin A, which is already in use against cancer, acne, cystic fibrosis, rheumatoid arthritis, and psoriasis, could competitively block RBP4 transport of vitamin A, a precursor LB from the blood to the RPE. See Radu R A, et al. (2005) Reductions in Serum Vitamin A Arrest Accumulation of Toxic Retinal Fluorophores: A Potential Therapy for Treatment of Lipofuscin-Based Retinal Diseases. Invest Ophthalmol Vis Sci 46(12):4393-4401. However, because vitamin A is a precursor of 11 cis retinal, a key molecule involved in normal vision, oral fenretinide showed side effects such as night blindness, and mild reversible skin-dryness. Although fenretinide appears to be effective at slowing down the formation of new LB in animals, it has no effect on pre-existing LB deposits, which may explain why it did not confer a significant therapeutic effect in patients already diagnosed with Stargardt or AMD.

Oral emixustat hydrochloride (Acucela Inc) is a synthetic non-retinoid reversible inhibitor of the RPE65 enzyme, which converts all-trans-retinyl to 11-cis-retinal, promoting the endogenous synthesis of the latter. In May 2016, the results from the phase 2b/3 SEATTLE study did not show any significant difference in retinal degenerative rate or visual acuity changes. Emixustant also caused significant night blindness that limits it use. Like fenretinide, emixustat has no effect on pre-existing LB deposits.

Oral deuterated vitamin A (ALK-001), is vitamin A modified by replacing hydrogen with deuterium, a safe, non-radioactive isotope. Deuterated vitamin A has lower tendency to spontaneously dimerize into LB. Long-term, oral administration of ALK-001 to ABCA4−/−reduced the accumulation of lipofuscin and A2E by 70% and 80%, respectively. Assessment of the retina electric response to light signals (electroretinogram) revealed that ALK-001 treatment prevented the gradual loss of visual function observed in the ABCA4−/−mouse model. Safety phase-I clinical trials have been completed (NCT02230228) and Phase 2 multicenter clinical trial for the treatment of STGD1 (NCT02402660) are ongoing. Given that ALK-001 blocks formation of lipofuscin, it is uncertain as to whether it would have an effect on pre-existing LB deposits in AMD patients.

Oral aldehyde traps (VM200, Vision Medicines) constitute new drugs that react with retinaldehydes forming reversible Schiff bases and thus reducing the available levels of free aldehydes with cellular amine groups. See Maeda A, et al. (2012) Primary amines protect against retinal degeneration in mouse models of retinopathies. Nat Chem Biol 8(2):170-8.

Successful elimination of lipofuscin from RPE cells in monkey retinas after one year of oral administration of soprazan (a proton potassium-competitive acid-blocker) was reported. See Julien and Schraermeyer (2012) Lipofuscin can be eliminated from the retinal pigment epithelium of monkeys. Neurobiol Aging 33(10):2390-7.

Since LB deposits are refractory to degradation by lysosomal hydrolases, several groups have searched for exogenous enzymes with LB destroying activity, like Horseradish peroxidase (HRP). See, e.g., Wu Y, Zhou J, Fishkin N, Rittmann B E, Sparrow J R, Enzymatic degradation of A2E, a retinal pigment epithelial lipofuscin bisretinoid. J Am Chem Soc 133(4):849-57 (2011); Sparrow J R, Zhou J, Ghosh S K, Liu Z, Bisretinoid degradation and the ubiquitin-proteasome system. Adv Exp Med Biol 801:593-600 (2014); Yogalingam G, et al. Cellular uptake and delivery of Myeloperoxidase to lysosomes promotes lipofuscin degradation and lysosomal stress in retinal cells. J Biol Chem 292:4255-4265 (2017). However, the subproducts of the degradation by these enzymes are very toxic and can potentially induce severe retinal damage.

### Therapeutic Methods of the Present Technology

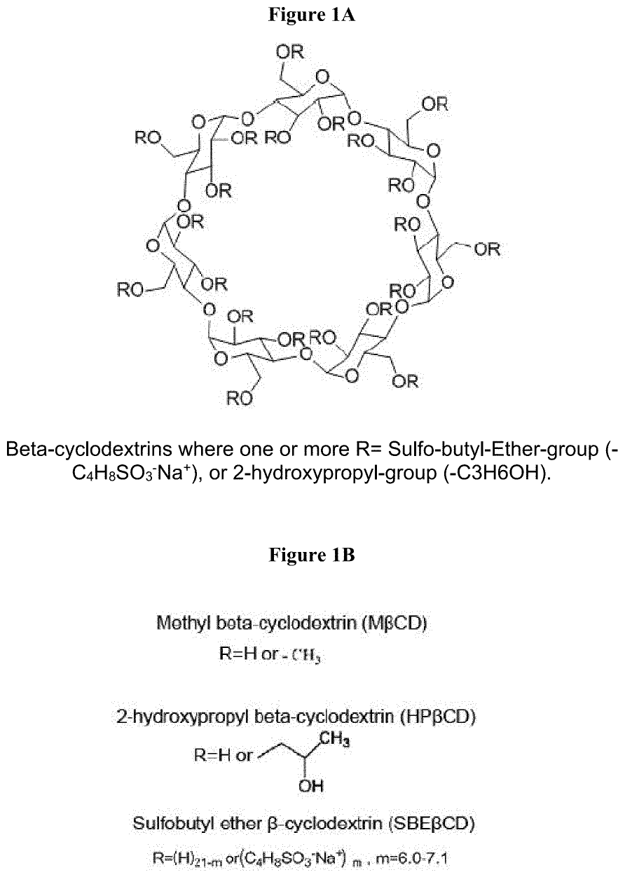

The present disclosure provides compositions that sequester and eliminate LB deposits from retinal cells, e.g., methyl beta-Cyclodextrin (MβCD), 2-hydroxypropyl beta-Cyclodextrin (HPβCD), sulfobutyl ether β-Cyclodextrin (SBEβCD), and any pharmaceutically acceptable salt thereof. See FIGS. 1A-1B. In one aspect, the present disclosure provides methods of preventing or treating a subject suffering from an eye disease associated with retinal cell lipofuscin accumulation, the method comprising administering to the subject a therapeutically effective amount of a substituted β-Cyclodextrin, wherein the substituted β-Cyclodextrin comprises a randomly substituted beta-Cyclodextrin with a degree of substitution (DS) between 4 and 14.5.

In one aspect, the present technology provides a method for preventing or treating an eye disease associated with retinal cell lipofuscin accumulation without impairing visual acuity in a subject in need thereof comprising administering to the subject an effective amount of sulfobutyl ether β-Cyclodextrin (SBE-βCD) or a pharmaceutically acceptable salt thereof. The eye disease associated with retinal cell lipofuscin accumulation may be selected from the group consisting of Stargardt disease (STGD), retinitis pigmentosa (RP), Age-Related Macular Degeneration (AMD), Best disease (BD), and cone-rod dystrophy. Conditions and diseases treatable by the methods described herein include any ophthalmologic or retinal disease or condition directly or indirectly caused by the accumulation of lipofuscin in retinal pigment epithelium (RPE) cells, which may be genetic or non-genetic, such as age-related macular degeneration (AMD), Stargardt disease, Best disease (BD), retinitis pigmentosa, and cone-rod dystrophy. Additionally or alternatively, in some embodiments, the effective amount of the β-cyclodextrin (e.g., SBEβCD) does not impair visual acuity when administered to the subject.

Additionally or alternatively, in some embodiments, the eye disease is genetic, non-genetic, or associated with aging. In another aspect, the present technology provides a method for preventing or treating retinal cell lipofuscin accumulation without impairing visual acuity in a subject in need thereof comprising administering to the subject an effective amount of sulfobutyl ether β-Cyclodextrin (SBE-βCD) or a pharmaceutically acceptable salt thereof.

Additionally or alternatively, in some embodiments of the methods disclosed herein, administration of the effective amount of the SBE-βCD or pharmaceutically acceptable salt thereof prevents exacerbation of lipofuscin-associated retinal damage in the subject.

Also disclosed herein are methods for reducing lipofuscin accumulation in retinal pigment epithelium cells comprising contacting the retinal pigment epithelium cells with an effective amount of sulfobutyl ether β-Cyclodextrin (SBE-βCD) or a pharmaceutically acceptable salt thereof.

Generally, the treatment considered herein has the effect of stopping, mitigating, or reversing the accumulation of lipofuscin bisretinoid lipid in RPE cells, and likewise, stopping, mitigating, or reversing the lipofuscin-associated damage or associated disease or condition. In some embodiments, the one or more β-cyclodextrins or pharmaceutically acceptable salts thereof are configured to complex and remove lipofuscin bisretinoid lipid in RPE cells. Without wishing to be bound by theory, it is believed that the one or more β-cyclodextrins or pharmaceutically acceptable salts thereof possess a cavity (i.e., binding pocket) suitable for accepting at least one lipofuscin bisretinoid lipid molecule as a partner. The result is a complex between the β-cyclodextrin(s) and lipofuscin bisretinoid lipid molecule. The interaction between the β-cyclodextrin(s) and lipofuscin bisretinoid lipid molecule is generally of a non-covalent nature, such as by hydrogen-bonding and/or van der Waals (dispersion) forces. The lipofuscin bisretinoid lipid is generally A2E, an A2E isomer, an oxidized derivative of A2E, A2-dihydropyridine-phosphatidylethanolamine, or an all-trans retinal dimer.

The term “pharmaceutically acceptable salt” means a salt prepared from a base or an acid which is acceptable for administration to a patient, such as a mammal (e.g., salts having acceptable mammalian safety for a given dosage regime). However, it is understood that the salts are not required to be pharmaceutically acceptable salts, such as salts of intermediate compounds that are not intended for administration to a patient. Pharmaceutically acceptable salts can be derived from pharmaceutically acceptable inorganic or organic bases and from pharmaceutically acceptable inorganic or organic acids. In addition, when one or more β-cyclodextrins of the present technology contains both a basic moiety, such as an amine, pyridine or imidazole, and an acidic moiety such as a carboxylic acid or tetrazole, zwitterions may be formed and are included within the term “salt” as used herein.

In one embodiment, the one or more β-cyclodextrins may contain one or more basic functional groups, such as amino or alkylamino, and thereby, can form pharmaceutically-acceptable salts by reaction with a pharmaceutically-acceptable acid. These salts can be prepared in situ in the administration vehicle or the dosage form manufacturing process, or by separately reacting a purified compound of the present technology in its free base form with a suitable organic or inorganic acid, and isolating the salt thus formed during subsequent purification. In another embodiment, the one or more β-cyclodextrins may contain one or more acidic functional groups, and thereby, can form pharmaceutically-acceptable salts by reaction with a pharmaceutically-acceptable base. These salts can likewise be prepared in situ in the administration vehicle or the dosage form manufacturing process, or by separately reacting the purified compound in its free acid form (e.g., hydroxyl or carboxyl) with a suitable base, and isolating the salt thus formed during subsequent purification.

Salts derived from pharmaceutically acceptable inorganic bases include ammonium, aluminum, calcium, copper, ferric, ferrous, lithium, magnesium, manganic, manganous, potassium, sodium, and zinc salts, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′-dibenzylethylenediamine, ethylamine, diethylamine, 2-diethylaminoethanol, 2-dimethylaminoethanol, ethanolamine, diethanolamine, ethylenediamine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine, trimethylamine, tripropylamine, tromethamine and the like. Salts derived from pharmaceutically acceptable inorganic acids include salts of boric, carbonic, hydrohalic (hydrobromic, hydrochloric, hydrofluoric or hydroiodic), nitric, phosphoric, sulfamic and sulfuric acids. Salts derived from pharmaceutically acceptable organic acids include salts of aliphatic hydroxyl acids (e.g., citric, gluconic, glycolic, lactic, lactobionic, malic, and tartaric acids), aliphatic monocarboxylic acids (e.g., acetic, butyric, formic, propionic and trifluoroacetic acids), amino acids (e.g., aspartic and glutamic acids), aromatic carboxylic acids (e.g., benzoic, 2-acetoxybenzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucuronic, mandelic, mucic, nicotinic, orotic, pamoic, pantothenic, sulfonic acids (e.g., benzenesulfonic, camphosulfonic, edisylic, ethanesulfonic, isethionic, methanesulfonic, naphthalenesulfonic, naphthalene-1,5-disulfonic, naphthalene-2,6-disulfonic and p-toluenesulfonic acids), xinafoic acid, valeric, oleic, palmitic, stearic, lauric, toluenesulfonic, methansulfonic, ethanedisulfonic, citric, ascorbic, maleic, oxalic, fumaric, phenylacetic, isothionic, succinic, tartaric, glutamic, salicylic, sulfanilic, napthylic, lactobionic, gluconic, laurylsulfonic acids, and the like.

Additionally or alternatively, in some embodiments, administration of the effective amount of the one or more β-cyclodextrins or pharmaceutically acceptable salts thereof prevent exacerbation of lipofuscin-associated retinal damage in the subject. In any and all embodiments of the methods disclosed herein, administration of the effective amount of the one or more β-cyclodextrins or pharmaceutically acceptable salts thereof block, mitigate, or reverse accumulation of lipofuscin in retinal pigment epithelium cells. In some embodiments of the methods disclosed herein, administration of the effective amount of the one or more β-cyclodextrins or pharmaceutically acceptable salts thereof prevent, slow the onset, or lessen the severity of lipofuscin-associated damage or a disease or condition directly or indirectly associated with lipofuscin-associated damage or lipofuscin accumulation in RPE cells of the subject. The subject can be of any gender (e.g., male or female), and/or can also be any age, such as elderly (generally, at least or above 60, 70, or 80 years of age), elderly-to-adult transition age subjects, adults, adult-to-pre-adult transition age subjects, and pre-adults, including adolescents (e.g., 13 and up to 16, 17, 18, or 19 years of age), children (generally, under 13 or before the onset of puberty), and infants. The subject can also be of any ethnic population or genotype. Some examples of human ethnic populations include Caucasians, Asians, Hispanics, Africans, African Americans, Native Americans, Semites, and Pacific Islanders.

Additionally or alternatively, in some embodiments, the one or more β-cyclodextrins or pharmaceutically acceptable salts thereof are configured to localize to RPE cells. In certain embodiments of the methods disclosed herein, the one or more β-cyclodextrins or pharmaceutically acceptable salts thereof are configured to complex with lipofuscin bisretinoid lipids in the RPE cells. The complex can be considered an organized chemical entity resulting from the association of two or more components held together by non-covalent intermolecular forces.

Additionally or alternatively, in certain embodiments, the one or more β-cyclodextrins or pharmaceutically acceptable salts thereof localize to RPE cells by being administered directly at, into, or in the adjacent vicinity of RPE cells, such as by injection or implantation. In other embodiments, the one or more β-cyclodextrins or pharmaceutically acceptable salts thereof localize to RPE cells by coupling the one or more j-cyclodextrins or pharmaceutically acceptable salts thereof with a targeting agent that selectively targets RPE cells, and the one or more β-cyclodextrins or pharmaceutically acceptable salts thereof may be administered at, into, or in the adjacent vicinity of RPE cells, or remotely from the RPE cells (e.g., by systemic administration). The cell-targeting agent (i.e., “targeting agent”) is any chemical entity that has the ability to bind to (i.e., “target”) a RPE cell. The cell-targeting agent may target any part of the RPE cell, e.g., cell membrane, organelle (e.g., lysosome or endosome), or cytoplasm. In one embodiment, the cell-targeting agent targets a component of a RPE cell in a selective manner. By selectively targeting a component of an RPE cell, the cell-targeting agent can, for example, selectively target certain components of cells over other types of cellular components. In other embodiments, the targeting agent targets cellular components non-selectively, e.g., by targeting cellular components found in most or all cells.

In various embodiments, the targeting agent can be, or include, for example, a peptide, dipeptide, tripeptide (e.g., glutathione), tetrapeptide, pentapeptide, hexapeptide, higher oligopeptide, protein, monosaccharide, disaccharide, trisaccharide, tetrasaccharide, higher oligosaccharide, polysaccharide (e.g., a carbohydrate), nucleobase, nucleoside (e.g., adenosine, cytidine, uridine, guanosine, thymidine, inosine, and S-Adenosyl methionine), nucleotide (i.e., mono-, di-, or tri-phosphate forms), dinucleotide, trinucleotide, tetranucleotide, higher oligonucleotide, nucleic acid, cofactor (e.g., TPP, FAD, NAD, coenzyme A, biotin, lipoamide, metal ions (e.g., Mg²⁺), metal-containing clusters (e.g., the iron-sulfur clusters), or a non-biological (i.e., synthetic) targeting group. Some particular types of proteins include enzymes, hormones, antibodies (e.g., monoclonal antibodies), lectins, and steroids.

Antibodies for use as targeting agents are generally specific for one or more cell surface antigens. In a particular embodiment, the antigen is a receptor. The antibody can be a whole antibody, or alternatively, a fragment of an antibody that retains the recognition portion (i.e., hypervariable region) of the antibody. Some examples of antibody fragments include Fab, Fe, and F(ab′)₂. In particular embodiments, particularly for the purpose of facilitating crosslinking of the antibody to the one or more β-cyclodextrins or pharmaceutically acceptable salts thereof described herein, the antibody or antibody fragment can be chemically reduced to derivatize the antibody or antibody fragment with sulfhydryl groups. In certain embodiments, the targeting agent is a ligand of an internalized receptor of the target cell. For example, the targeting agent can be a targeting signal for acid hydrolase precursor proteins that transport various materials to lysosomes. One such targeting agent of particular interest is mannose-6-phosphate (M6P), which is recognized by mannose 6-phosphate receptor (MPR) proteins in the trans-Golgi. Endosomes are known to be involved in transporting M6P-labeled substances to lysosomes.

In other embodiments, the targeting agent is a peptide containing an RGD sequence, or variants thereof, that bind RGD receptors on the surface of many types of cells. Other targeting agents include, for example, transferrin, insulin, amylin, and the like. Receptor internalization may be used to facilitate intracellular delivery of the one or more 3-cyclodextrins or pharmaceutically acceptable salts thereof described herein. In certain embodiments, one cell-targeting molecule or group, or several (e.g., two, three, or more) of the same type of cell-targeting molecule or group are attached to the one or more 3-cyclodextrins or pharmaceutically acceptable salts thereof directly or via a linker. In other embodiments, two or more different types of targeting molecules are attached to the one or more β-cyclodextrins or pharmaceutically acceptable salts thereof directly or via a linker.

Additionally or alternatively, in some embodiments, a fluorophore may be attached to the one or more β-cyclodextrins or pharmaceutically acceptable salts thereof. Incorporation of one or more fluorophores can have several purposes. In some embodiments, one or more fluorophores are included in order to quantify cellular uptake and retention of the one or more β-cyclodextrins or pharmaceutically acceptable salts thereof (e.g., by a fluorescence spectroscopic method).

As used herein, a “fluorophore” refers to any species with the ability to fluoresce (i.e., that possesses a fluorescent property). For example, in one embodiment, the fluorophore is an organic fluorophore. The organic fluorophore can be, for example, a charged (i.e., ionic) molecule (e.g., sulfonate or ammonium groups), uncharged (i.e., neutral) molecule, saturated molecule, unsaturated molecule, cyclic molecule, bicyclic molecule, tricyclic molecule, polycyclic molecule, acyclic molecule, aromatic molecule, and/or heterocyclic molecule (i.e., by being ring-substituted by one or more heteroatoms selected from, for example, nitrogen, oxygen and sulfur). In the particular case of unsaturated fluorophores, the fluorophore contains one, two, three, or more carbon-carbon and/or carbon-nitrogen double and/or triple bonds. In a particular embodiment, the fluorophore contains at least two (e.g., two, three, four, five, or more) conjugated double bonds aside from any aromatic group that may be in the fluorophore. In other embodiments, the fluorophore is a fused polycyclic aromatic hydrocarbon (PAH) containing at least two, three, four, five, or six rings (e.g., naphthalene, pyrene, anthracene, chrysene, triphenylene, tetracene, azulene, and phenanthrene) wherein the PAH can be optionally ring-substituted or derivatized by one, two, three or more heteroatoms or heteroatom-containing groups.

In other embodiments, the organic fluorophore is a xanthene derivative (e.g., fluorescein, rhodamine, Oregon green, eosin, and Texas Red), cyanine or its derivatives or subclasses (e.g., streptocyanines, hemicyanines, closed chain cyanines, phycocyanins, allophycocyanins, indocarbocyanines, oxacarbocyanines, thiacarbocyanines, merocyanins, and phthalocyanines), naphthalene derivatives (e.g., dansyl and prodan derivatives), coumarin and its derivatives, oxadiazole and its derivatives (e.g., pyridyloxazoles, nitrobenzoxadiazoles, and benzoxadiazoles), pyrene and its derivatives, oxazine and its derivatives (e.g., Nile Red, Nile Blue, and cresyl violet), acridine derivatives (e.g., proflavin, acridine orange, and acridine yellow), arylmethine derivatives (e.g., auramine, crystal violet, and malachite green), and tetrapyrrole derivatives (e.g., porphyrins and bilirubins). Some particular families of dyes considered herein are the Cy® family of dyes, the Alexa® family of dyes, the ATTO® family of dyes, and the Dy® family of dyes. The ATTO® dyes, in particular, can have several structural motifs, including, coumarin-based, rhodamine-based, carbopyronin-based, and oxazine-based structural motifs.

The fluorophore can be attached to the one or more β-cyclodextrins or pharmaceutically acceptable salts thereof by any of the linking methodologies known in the art. For example, a commercial mono-reactive fluorophore (e.g., NHS-Cy5) or bis-reactive fluorophore (e.g., bis-NHS-Cy5 or bis-maleimide-Cy5) can be used to link the fluorophore to one or more molecules containing appropriate reactive groups (e.g., amino, thiol, hydroxy, aldehydic, or ketonic groups). Alternatively, the one or more β-cyclodextrins or pharmaceutically acceptable salts thereof can be derivatized with one, two, or more such reactive groups, and these reactive portions reacted with a fluorophore containing appropriate reactive groups (e.g., an amino-containing fluorophore).

The one or more β-cyclodextrins or pharmaceutically acceptable salts thereof can be administered by any route that permits contact with RPE cells. The administration can be, for example, ocular, parenteral (e.g., subcutaneous, intramuscular, or intravenous), topical, transdermal, intravitreous, retro-orbital, subretinal, subscleral, oral, sublingual, or buccal modes of administration. Some of the foregoing exemplary modes of administration can be achieved by injection. However, in some embodiments, injection is avoided by use of a slow-release implant in the vicinity of the retina (e.g., subscleral route) or by administering drops to the conjuctiva. The one or more R-cyclodextrins or pharmaceutically acceptable salts thereof of the present technology may be administered locally, to the eyes of patients suffering from lipofuscin accumulation including Stargardt, carriers of ABCA4 defective genes, dry AMD or at risk for developing retinal degeneration due to the accumulation of lipid bisretinoids (lipofuscin). Local administration includes intravitreal, topical ocular, transdermal patch, subdermal, parenteral, intraocular, subconjunctival, or retrobulbar or subtenon's injection, trans-scleral (including iontophoresis), posterior juxtascleral delivery, or slow release biodegradable polymers or liposomes. The one or more β-cyclodextrins or pharmaceutically acceptable salts thereof can also be delivered in ocular irrigating solutions. Concentrations may range from about 0.001 μM to about 100 μM, preferably about 0.01 μM to about 5 μM.

In some embodiments, the one or more β-cyclodextrins or pharmaceutically acceptable salts thereof are administered, at least initially, at levels lower than that required in order to achieve a desired therapeutic effect, and the dose is gradually or suddenly increased until a desired effect is achieved. In other embodiments, the one or more β-cyclodextrins or pharmaceutically acceptable salts thereof are administered, at least initially, at levels higher than that required in order to accelerate a desired therapeutic effect, and the dose gradually or suddenly moderated until a desired effect is achieved.

The selected dosage level will depend upon several factors, as determined by a medical practitioner. Some of these factors include the type of disease or condition being treated, the stage or severity of the condition or disease, the efficacy of the therapeutic compound being used and its bioavailability profile, as well as the specifics (e.g., genotype and phenotype) of the subject being treated, e.g., age, sex, weight, and overall condition.

Particularly for systemic modes of administration, the dosage can be, for example, in the range of about 0.01, 0.1, 0.5, 1, 5, or 10 mg per kg of body weight per day to about 20, 50, 100, 500, or 1000 mg per kilogram of body weight per day, or bi-daily, or twice, three, four, or more times a day. Particularly in embodiments where the one or more β-cyclodextrins or pharmaceutically acceptable salts thereof are administered non-systemically directly at the retina, the dosage can disregard body weight, and can be in smaller amounts (e.g., 1-1000 μg per dose). In some embodiments, the daily dose of the one or more β-cyclodextrins or pharmaceutically acceptable salts thereof is the lowest dose effective to produce a therapeutic effect. In some embodiments, the one or more β-cyclodextrins or pharmaceutically acceptable salts thereof are not administered in discrete dosages, but in a continuous mode, such as provided by a slow release implant or intravenous line.

In one aspect, the present disclosure provides pharmaceutical compositions comprising a substituted β-Cyclodextrin, wherein the substituted β-Cyclodextrin comprises a randomly substituted beta-Cyclodextrin with a degree of substitution (DS) between 4 and 14.5. In one aspect, the present disclosure provides pharmaceutical compositions comprising one or more cyclodextrins selected from the group consisting of methyl beta-Cyclodextrin (MβCD), 2-hydroxypropyl beta-Cyclodextrin (HPβCD), sulfobutyl ether β-Cyclodextrin (SBEβCD), and pharmaceutically acceptable salts thereof.

The one or more β-cyclodextrins or pharmaceutically acceptable salts thereof may be formulated together with one or more pharmaceutically acceptable carriers (additives) and/or diluents, known in the art. The pharmaceutical compositions of the present technology may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) sublingually; (5) ocularly; (6) transdermally; or (7) nasally.

In some embodiments, pharmaceutical compositions of the present technology may contain one or more “pharmaceutically-acceptable carriers,” which as used herein, generally refers to a pharmaceutically-acceptable composition, such as a liquid or solid filler, diluent, excipient, manufacturing aid (e.g., lubricant, tale magnesium, calcium or zinc stearate, or steric acid), or solvent encapsulating material, useful for introducing the active agent into the body. Each carrier must be “acceptable” in the sense of being compatible with other ingredients of the formulation and not injurious to the patient. Examples of suitable aqueous and non-aqueous carriers that may be employed in the pharmaceutical compositions of the present technology include, for example, water, ethanol, polyols (such as glycerol, propylene glycol, polyethylene glycol, and the like), vegetable oils (such as olive oil), and injectable organic esters (such as ethyl oleate), and suitable mixtures thereof. Proper fluidity can be maintained, for example, by the use of coating materials, such as lecithin, by the maintenance of the required particle size in the case of dispersions, and by the use of surfactants.

In some embodiments, the formulations may include one or more of sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; alginic acid; buffering agents, such as magnesium hydroxide and aluminum hydroxide; pyrogen-free water; isotonic saline; Ringer's solution; ethyl alcohol; pH buffered solutions; polyesters, polycarbonates and/or polyanhydrides; preservatives; glidants; fillers; and other non-toxic compatible substances employed in pharmaceutical formulations.

Various auxiliary agents, such as wetting agents, emulsifiers, lubricants (e.g., sodium lauryl sulfate and magnesium stearate), coloring agents, release agents, coating agents, sweetening agents, flavoring agents, preservative agents, and antioxidants can also be included in the pharmaceutical composition. Some examples of pharmaceutically-acceptable antioxidants include: (1) water soluble antioxidants, such as ascorbic acid, cysteine hydrochloride, sodium bisulfate, sodium metabisulfite, sodium sulfite, and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like. In some embodiments, the pharmaceutical formulation includes an excipient selected from, for example, celluloses, liposomes, micelle-forming agents (e.g., bile acids), and polymeric carriers, e.g., polyesters and polyanhydrides. Suspensions, in addition to the active compounds, may contain suspending agents, such as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof. Prevention of the action of microorganisms on the active compounds may be ensured by the inclusion of various antibacterial and antifungal agents, such as, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption, such as aluminum monostearate and gelatin.

Pharmaceutical formulations of the present technology may be prepared by any of the methods known in the pharmaceutical arts. The amount of active ingredient that can be combined with a carrier material to produce a single dosage form will vary depending upon the host being treated and the particular mode of administration. The amount of active ingredient that can be combined with a carrier material to produce a single dosage form will generally be that amount of the compound that produces a therapeutic effect. Generally, the amount of active compound will be in the range of about 0.1 to 99 percent, more typically, about 5 to 70 percent, and more typically, about 10 to 30 percent.

The compositions of the present technology may be administered locally, to the eyes of patients suffering from lipofuscin accumulation including Stargardt, carriers of ABCA4 defective genes, dry AMD or at risk for developing retinal degeneration due to the accumulation of lipid bisretinoids (lipofuscin). The one or more β-cyclodextrins of the present technology or pharmaceutically acceptable salts thereof can be incorporated into various types of ophthalmic formulations for delivery to the eye (e.g., topically, intracamerally, juxtasclerally, or via an implant). The one or more β-cyclodextrins of the present technology or pharmaceutically acceptable salts thereof may be combined with ophthalmologically acceptable preservatives, surfactants, viscosity enhancers, gelling agents, penetration enhancers, buffers, sodium chloride, and water to form aqueous, sterile ophthalmic suspensions or solutions or preformed gels or gels formed in situ.

In some embodiments, the substituted β-Cyclodextrin of the present technology (e.g., methyl beta-Cyclodextrin (MβCD), 2-hydroxypropyl beta-Cyclodextrin (HPβCD), sulfobutyl ether β-Cyclodextrin (SBEβCD), and pharmaceutically acceptable salts thereof) is administered 1-10 times a day, once a day, twice, three, four, or more times a day, 1-3 times a day, 2-4 times a day, 3-6 times a day, 4-8 times a day or 5-10 times a day. In some embodiments, the substituted β-Cyclodextrin (e.g., methyl beta-Cyclodextrin (MβCD), 2-hydroxypropyl beta-Cyclodextrin (HPβCD), sulfobutyl ether β-Cyclodextrin (SBEβCD), and pharmaceutically acceptable salts thereof) is administered every day, every other day, 2-3 times a week, or 3-6 times a week.

In some embodiments, the dose of the substituted β-Cyclodextrin (e.g., methyl beta-Cyclodextrin (MβCD), 2-hydroxypropyl beta-Cyclodextrin (HPβCD), sulfobutyl ether β-Cyclodextrin (SBEβCD), and pharmaceutically acceptable salts thereof) can be, for example, in the range of about 0.01, 0.1, 0.5, 1, 5, 10, or 100 mg per kg of body weight per day to about 20, 50, 100, 500, or 1000 mg per kilogram of body weight. Particularly in embodiments where the active substance is administered directly at the retina, the dosage administered can be independent of body weight, and can be in smaller amounts (e.g., 1-1000 μg per dose).

If dosed topically, the one or more β-cyclodextrins of the present technology or pharmaceutically acceptable salts thereof may be formulated as topical ophthalmic suspensions or solutions, with a pH of about 4 to 8. The one or more β-cyclodextrins of the present technology or pharmaceutically acceptable salts thereof will normally be contained in these formulations in an amount 0.001% to 5% by weight, or in an amount of 0.01% to 2% by weight. Thus, for topical presentation, 1 to 2 drops of these formulations would be delivered to the surface of the eye 1 to 4 times per day according to the discretion of a skilled clinician. In some embodiments, the pharmaceutical compositions of the present technology, containing therapeutically effective amounts of at least one monomeric or polymeric cyclodextrins, are delivered intravitreally either through an injection (perhaps microspheres), an intravitreal device, or placed in the sub-Tenon space by injection, gel, or implant, or by other methods discussed above. If delivered as a solution, the therapeutically effective amount of the one or more β-cyclodextrins of the present technology or pharmaceutically acceptable salts thereof in the composition might be about 18-44 μM, of a concentration of about 20-50%. If formulated as a suspension, a therapeutically effective amount of the one or more 3-cyclodextrins of the present technology or pharmaceutically acceptable salts thereof is about 20-80%. In another embodiment, the therapeutically effective amount of the one or more β-cyclodextrins of the present technology or pharmaceutically acceptable salts thereof is administered in the form of a mini-tablet, each weighing from about 1 mg to about 40 mg, or about 5 mg. From one to twenty such mini-tablets may be injected [dry] into the sub-Tenon space through a trochar in one dose, so that a total single dose of 50-100 mg [44-88 μM] is injected.

In some embodiments, the substituted β-Cyclodextrin (e.g., methyl beta-Cyclodextrin (MβCD), 2-hydroxypropyl beta-Cyclodextrin (HPβCD), sulfobutyl ether β-Cyclodextrin (SBEβCD), and pharmaceutically acceptable salts thereof) is administered 1-10 times a day, once a day, twice, three, four, or more times a day, 1-3 times a day, 2-4 times a day, 3-6 times a day, 4-8 times a day or 5-10 times a day. In some embodiments, the substituted β-Cyclodextrin (e.g., methyl beta-Cyclodextrin (MβCD), 2-hydroxypropyl beta-Cyclodextrin (HPβCD), sulfobutyl ether β-Cyclodextrin (SBEβCD), and pharmaceutically acceptable salts thereof) is administered every day, every other day, 2-3 times a week, or 3-6 times a week.

In some embodiments, the dose of the substituted β-Cyclodextrin (e.g., methyl beta-Cyclodextrin (MβCD), 2-hydroxypropyl beta-Cyclodextrin (HPβCD), sulfobutyl ether β-Cyclodextrin (SBEβCD), and pharmaceutically acceptable salts thereof) can be, for example, in the range of about 0.01, 0.1, 0.5, 1, 5, 10, or 100 mg per kg of body weight per day to about 20, 50, 100, 500, or 1000 mg per kilogram of body weight. Particularly in embodiments where the active substance is administered directly at the retina, the dosage administered can be independent of body weight, and can be in smaller amounts (e.g., 1-1000 μg per dose).

Formulations of the present technology suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present technology as an active ingredient. The active compound may also be administered as a bolus, electuary, or paste.

Methods of preparing these formulations generally include the step of admixing a 3-cyclodextrin of the present technology or pharmaceutically acceptable salt thereof, with the carrier, and optionally, one or more auxiliary agents. In the case of a solid dosage form (e.g., capsules, tablets, pills, powders, granules, trouches, and the like), the active compound can be admixed with a finely divided solid carrier, and typically, shaped, such as by pelletizing, tableting, granulating, powderizing, or coating. Generally, the solid carrier may include, for example, sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and/or (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more auxiliary ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. The tablets, and other solid dosage forms of the active agent, such as capsules, pills and granules, may optionally be scored or prepared with coatings and shells, such as enteric coatings and other coatings well known in the pharmaceutical-formulating art. The dosage form may also be formulated so as to provide slow or controlled release of the active ingredient therein using, for example, hydroxypropylmethyl cellulose in varying proportions to provide the desired release profile, other polymer matrices, liposomes and/or microspheres. The dosage form may alternatively be formulated for rapid release, e.g., freeze-dried.

Generally, the dosage form is required to be sterile. For this purpose, the dosage form may be sterilized by, for example, filtration through a bacteria-retaining filter, or by incorporating sterilizing agents in the form of sterile solid compositions which can be dissolved in sterile water, or some other sterile injectable medium immediately before use. The pharmaceutical compositions may also contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms are typically a pharmaceutically acceptable emulsion, microemulsion, solution, suspension, syrup, or elixir of the active agent. In addition to the active ingredient, the liquid dosage form may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Dosage forms specifically intended for topical or transdermal administration can be in the form of, for example, a powder, spray, ointment, paste, cream, lotion, gel, solution, or patch. Ophthalmic formulations, such as eye ointments, powders, solutions, and the like, are also contemplated herein. The active compound may be mixed under sterile conditions with a pharmaceutically-acceptable carrier, and with any preservatives, buffers, or propellants that may be required. The topical or transdermal dosage form may contain, in addition to an active compound of this present technology, one or more excipients, such as those selected from animal and vegetable fats, oils, waxes, paraffins, starch, tragacanth, cellulose derivatives, polyethylene glycols, silicones, bentonites, silicic acid, talc and zinc oxide, and mixtures thereof. Sprays may also contain customary propellants, such as chlorofluorohydrocarbons and volatile unsubstituted hydrocarbons, such as butane and propane.

For purposes of this present technology, transdermal patches may provide the advantage of permitting controlled delivery of a compound of the present technology into the body. Such dosage forms can be made by dissolving or dispersing the compound in a suitable medium. Absorption enhancers can also be included to increase the flux of the compound across the skin. The rate of such flux can be controlled by either providing a rate-controlling membrane or dispersing the compound in a polymer matrix or gel.

Pharmaceutical compositions of this present technology suitable for parenteral administration generally include one or more compounds of the present technology in combination with one or more pharmaceutically-acceptable sterile isotonic aqueous or nonaqueous solutions, dispersions, suspensions or emulsions, or sterile powders that may be reconstituted into sterile injectable solutions or dispersions prior to use, which may contain sugars, alcohols, antioxidants, buffers, bacteriostats, or solutes that render the formulation isotonic with the blood of the intended recipient.

In some cases, in order to prolong the effect of a drug, it may be desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms can be made by forming microencapsule matrices of the active compound in a biodegradable polymer, such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations can also be prepared by entrapping the drug in liposomes or microemulsions that are compatible with body tissue.

The pharmaceutical composition may also be in the form of a microemulsion. In the form of a microemulsion, bioavailability of the active agent may be improved. Reference is made to Dordunoo, S. K., et al., Drug Development and Industrial Pharmacy, 17(12), 1685-1713, 1991, and Sheen, P. C., et al., J. Pharm. Sci., 80(7), 712-714, 1991, the contents of which are herein incorporated by reference in their entirety.

The pharmaceutical composition may also contain micelles formed from a compound of the present technology and at least one amphiphilic carrier, in which the micelles have an average diameter of less than about 100 nm. In some embodiments, the micelles have an average diameter less than about 50 nm, or an average diameter less than about 30 nm, or an average diameter less than about 20 nm.

While any suitable amphiphilic carrier is considered herein, the amphiphilic carrier is generally one that has been granted Generally-Recognized-as-Safe (GRAS) status, and that can both solubilize the compound of the present technology and microemulsify it at a later stage when the solution comes into a contact with a complex water phase (such as one found in the living biological tissue). Usually, amphiphilic ingredients that satisfy these requirements have HLB (hydrophilic to lipophilic balance) values of 2-20, and their structures contain straight chain aliphatic radicals in the range of C-6 to C-20. Some examples of amphiphilic agents include polyethylene-glycolized fatty glycerides and polyethylene glycols.

Some amphiphilic carriers are saturated and monounsaturated polyethyleneglycolyzed fatty acid glycerides, such as those obtained from fully or partially hydrogenated various vegetable oils. Such oils may advantageously consist of tri-. di- and mono-fatty acid glycerides and di- and mono-polyethyleneglycol esters of the corresponding fatty acids, such as a fatty acid composition including capric acid 4-10, capric acid 3-9, lauric acid 40-50, myristic acid 14-24, palmitic acid 4-14 and stearic acid 5-15%. Another useful class of amphiphilic carriers includes partially esterified sorbitan and/or sorbitol, with saturated or mono-unsaturated fatty acids (SPAN-series) or corresponding ethoxylated analogs (TWEEN-series). Commercially available amphiphilic carriers are particularly contemplated, including the Gelucire®-series, Labrafil®, Labrasol®, or Lauroglycol®, PEG-mono-oleate, PEG-di-oleate, PEG-mono-laurate and di-laurate, Lecithin, Polysorbate 80.

Hydrophilic polymers suitable for use in the pharmaceutical composition are generally those that are readily water-soluble, can be covalently attached to a vesicle-forming lipid, and that are tolerated in vivo without substantial toxic effects (i.e., are biocompatible). Suitable polymers include, for example, polyethylene glycol (PEG), polylactic (also termed polylactide), polyglycolic acid (also termed polyglycolide), a polylactic-polyglycolic acid copolymer, and polyvinyl alcohol. Exemplary polymers are those having a molecular weight of from about 100 or 120 daltons up to about 5,000 or 10,000 daltons, and more preferably from about 300 daltons to about 5,000 daltons. In certain embodiments, the polymer is polyethylene glycol having a molecular weight of from about 100 to about 5,000 daltons, or a molecular weight of from about 300 to about 5,000 daltons, or a molecular weight of 750 daltons, i.e., PEG (750). Polymers may also be defined by the number of monomers therein. In some embodiments, the pharmaceutical compositions of the present technology utilize polymers of at least about three monomers, such PEG polymers comprising of at least three monomers, or approximately 150 daltons. Other hydrophilic polymers that may be suitable for use in the present technology include polyvinylpyrrolidone, polymethoxazoline, polyethyloxazoline, polyhydroxypropyl methacrylamide, polymethacrylamide, polydimethylacrylamide, and derivatized celluloses such as hydroxymethylcellulose or hydroxyethylcellulose.

In certain embodiments, the pharmaceutical composition includes a biocompatible polymer selected from polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, and copolymers thereof.

The pharmaceutical composition may also be in liposomal form. Liposomes contain at least one lipid bilayer membrane enclosing an aqueous internal compartment. Liposomes may be characterized by membrane type and by size. Small unilamellar vesicles (SUVs) have a single membrane and typically range from 0.02 to 0.05 μm in diameter; large unilamellar vesicles (LUVS) are typically larger than 0.05 am Oligolamellar large vesicles and multilamellar vesicles have multiple, usually concentric, membrane layers, and are typically larger than 0.1 μm. The liposomes may also contain several smaller vesicles contained within a larger vesicle, i.e., multivesicular vesicles.

In some embodiments, the pharmaceutical composition includes liposomes containing one or more β-cyclodextrins or pharmaceutically acceptable salts thereof of the present technology, where the liposome membrane is formulated to provide an increased carrying capacity. Alternatively or additionally, the one or more β-cyclodextrins or pharmaceutically acceptable salts of the present technology may be contained within, or adsorbed onto, the liposome bilayer of the liposome. In some embodiments, the active agent may be aggregated with a lipid surfactant and carried within the liposome's internal space. In such cases, the liposome membrane is formulated to resist the disruptive effects of the active agent-surfactant aggregate. In certain embodiments, the lipid bilayer of a liposome contains lipids derivatized with polyethylene glycol (PEG), such that the PEG chains extend from the inner surface of the lipid bilayer into the interior space encapsulated by the liposome, and extend from the exterior of the lipid bilayer into the surrounding environment.

Active agents contained within liposomes are preferably in solubilized form. Aggregates of surfactant and active agent (such as emulsions or micelles containing the active agent of interest) may be entrapped within the interior space of liposomes. A surfactant typically serves to disperse and solubilize the active agent. The surfactant may be selected from any suitable aliphatic, cycloaliphatic or aromatic surfactant, including but not limited to biocompatible lysophosphatidylcholines (LPCs) of varying chain lengths, e.g., from about 14 to 20 carbons. Polymer-derivatized lipids, such as PEG-lipids, may also be utilized for micelle formation as they will act to inhibit micelle/membrane fusion, and as the addition of a polymer to surfactant molecules decreases the critical micelle concentration (CMC) of the surfactant and aids in micelle formation. Preferred are surfactants with CMCs in the micromolar range; higher CMC surfactants may be utilized to prepare micelles entrapped within liposomes of the present technology, however, micelle surfactant monomers could affect liposome bilayer stability and would be a factor in designing a liposome of a desired stability.

Liposomes according to the present technology may be prepared by any of a variety of techniques known in the art, such as described in, for example, U.S. Pat. No. 4,235,871 and International Published Application WO 96/14057, the contents of which are incorporated herein by reference in their entirety. For example, liposomes may be prepared by diffusing a lipid derivatized with a hydrophilic polymer into preformed liposomes, such as by exposing preformed liposomes to micelles composed of lipid-grafted polymers, at lipid concentrations corresponding to the final mole percent of derivatized lipid which is desired in the liposome. Liposomes containing a hydrophilic polymer can also be formed by homogenization, lipid-field hydration, or extrusion techniques, as are known in the art. By another methodology, the active agent is first dispersed by sonication in a lysophosphatidylcholine or other low critical micelle concentration (CMC) surfactant (including polymer grafted lipids) that readily solubilizes hydrophobic molecules. The resulting micellar suspension of active agent is then used to rehydrate a dried lipid sample that contains a suitable mole percent of polymer-grafted lipid, or cholesterol. The lipid and active agent suspension is then formed into liposomes using extrusion techniques well known in the art, and the resulting liposomes separated from the unencapsulated solution by standard column separation.

In some embodiments, the liposomes are prepared to have substantially homogeneous sizes in a selected size range. One effective sizing method involves extruding an aqueous suspension of the liposomes through a series of polycarbonate membranes having a selected uniform pore size. The pore size of the membrane will correspond roughly with the largest sizes of liposomes produced by extrusion through the membrane (U.S. Pat. No. 4,737,323, the contents of which are herein incorporated by reference in their entirety).

The release characteristics of a formulation of the present technology depend on several factors, including, for example, the type and thickness of the encapsulating material, the concentration of encapsulated drug, and the presence of release modifiers. If desired, the release can be manipulated to be pH dependent, such as by using a pH-sensitive coating that releases only at a low pH, as in the stomach, or releases at a higher pH, as in the intestine. An enteric coating can be used to prevent release from occurring until after passage through the stomach. Multiple coatings or mixtures of cyanamide encapsulated in different materials can be used to obtain an initial release in the stomach, followed by later release in the intestine. Release can also be manipulated by inclusion of salts or pore-forming agents, which can increase water uptake or release of drug by diffusion from the capsule. Excipients that modify the solubility of the drug can also be used to control the release rate. Agents that enhance degradation of the matrix or release from the matrix can also be incorporated. The agents can be added to the drug, added as a separate phase (i.e., as particulates), or can be co-dissolved in the polymer phase depending on the compound. In all cases, the amount is preferably between 0.1 and thirty percent (w/w polymer). Some types of degradation enhancers include inorganic salts, such as ammonium sulfate and ammonium chloride; organic acids, such as citric acid, benzoic acid, and ascorbic acid; inorganic bases, such as sodium carbonate, potassium carbonate, calcium carbonate, zinc carbonate, and zinc hydroxide; organic bases, such as protamine sulfate, spermine, choline, ethanolamine, diethanolamine, and triethanolamine; and surfactants, such as a Tween™ or Pluronic™ commercial surfactant. Pore-forming agents that add microstructure to the matrices (i.e., water-soluble compounds, such as inorganic salts and sugars) are generally included as particulates.

Uptake can also be manipulated by altering residence time of the particles in the body. This can be achieved by, for example, coating the particle with, or selecting as the encapsulating material, a mucosal adhesive polymer. Examples include most polymers with free carboxyl groups, such as chitosan, celluloses, and especially polyacrylates (as used herein, polyacrylates refers to polymers including acrylate groups and modified acrylate groups such as cyanoacrylates and methacrylates).

### Examples

The present technology is further illustrated by the following Examples, which should not be construed as limiting in any way. The examples herein are provided to illustrate advantages of the present technology and to further assist a person of ordinary skill in the art with preparing or using the compositions and systems of the present technology. The examples should in no way be construed as limiting the scope of the present technology, as defined by the appended claims. The examples can include or incorporate any of the variations, aspects, or embodiments of the present technology described above. The variations, aspects, or embodiments described above may also further each include or incorporate the variations of any or all other variations, aspects or embodiments of the present technology.

### Example 1: Materials and Methods

Sensitive and quantitative methods to assess the amounts of lipofuscin bisretinoids in cells were developed based on the intrinsic auto-fluorescence of LBs.

Fluorescence microscopy: LBs are auto-fluorescent molecules on account of the abundant conjugated double bonds in their hydrophobic arms. When excited at 430 nm (blue light) they emit yellow-orange fluorescence peaking at 610 nm. The intensity of the fluorescence is proportional to their amounts. Hoechst blue fluorescence dye was used to stain the nucleus of cells treated with cyclodextrins. In this way, one is able to express the 610 nm emission peak (amount of LBs) in relationship to the number of cells in the imaged field. Each dose was assayed in triplicated and ten random fields were evaluated per well.

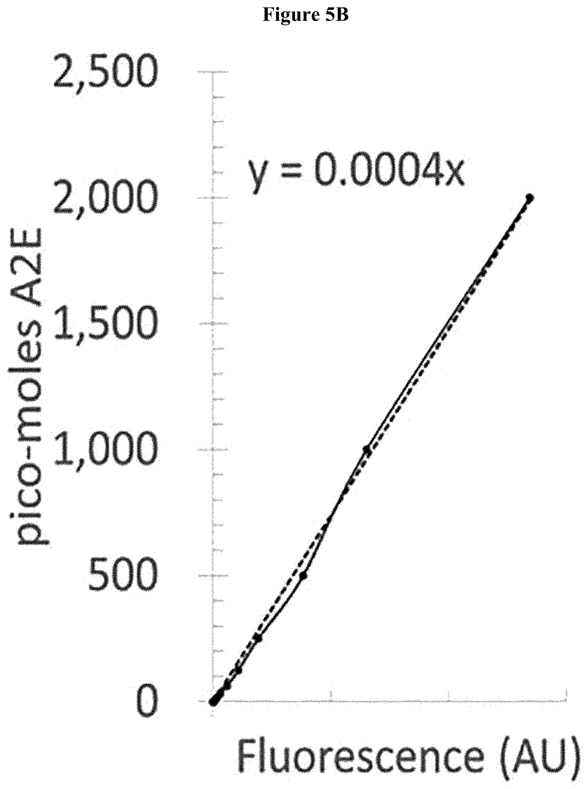

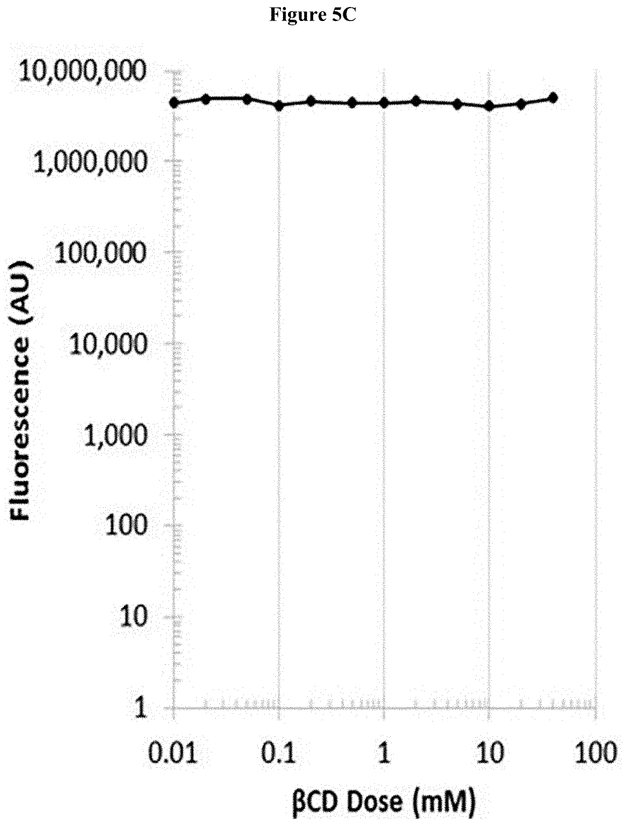

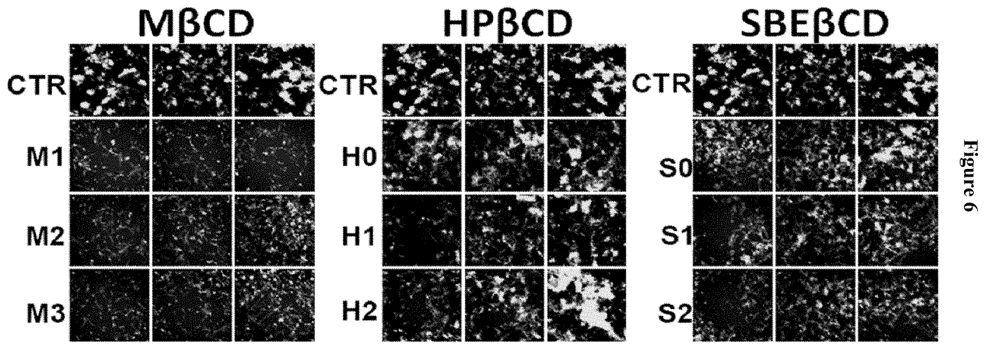

Fluorescence plate reader method: Methyl β-Cyclodextrins reduce lipid bisretinoids (LB) content, in vitro and in vivo, using fluorescence microscopy and HPLC (Nociari et al., Proc Natl Acad Sci USA. 111(14): E1402-E1408 (2014)). However, these methods lack sufficient linearity and sensitivity to provide robust and unbiased assessment of LB quantities. To overcome these limitations, a simple and sensitive microplate assay was developed to quantify LB removal. Briefly, the conditions to preload a confluent culture of RPE cells with non-toxic doses of A2E (5 μM) were optimized. The cells were preloaded with low doses of A2E in one or repetitive steps, at least one week before the assay. These cells may be kept loaded for months until needed for a removal assay. 48 hours before an assay, cells were then trypsinized, counted and seeded in 96 well glass bottom plates at a density of 1×10⁵ cells per well. After 48 hrs, cells were treated O/N (at least in quadruplicate) with the β-Cyclodextrin formulations (FIG. 5A). After treatment with β-Cyclodextrins, supernatants were removed and cells were collected with an A2E-extraction buffer to maximize A2E autofluorescence while preventing solvatochromic interferences by β-Cyclodextrin complexes (FIG. 5B), which yielded an extraordinary linearity between 5×10⁴ to 2×10³ pico-moles A2E (FIG. 5C).

Since fluorescence was used to determine A2E amount, and considering that A2E exhibits stronger fluorescence when complexed with cyclodextrins, an A2E-extraction buffer was developed that overcomes this interference and maximizes the fluorescent detection of A2E. The A2E-extraction buffer contains a mix of 2% Triton X-100 and 1% SDS in PBS. Triton-100 is known to form complexes with 3 Cyclodextrins at higher affinity than A2E. At 2% Triton, there is a great excess of detergent respect any amount of A2E that could be potentially be released from the loaded cells. This serves two purposes: (i) to force all A2E molecules out of the Cyclodextrin cavities; and (ii) to uniformly surround A2E with detergent molecules, thus shielding them against water quenching to increase intensity of emission, and consequently improving the sensitivity of the assay.

The cyclodextrins (CD) used in instant study are shown in Table 1. The degree of substitution in a CD molecule can vary from 1 to 21. DS is the average number of substituents per CD molecule. FIGS. 1A-1B show the structural formulae of the compositions of certain CD molecules of the present technology.

TABLE 1

| | Tested CDs | Company | Catalog # | Lot # | DS |
|---|---|---|---|---|---|
| A0 | α-Cyclodextrin | TCI America, | C0776 | TIJH-BB | 0 |
| | | Portland, OR | | | |
| B0 | β-Cyclodextrin | Cyclolab, Budapest, | CY-2009 | CYL-4264 | 0 |
| | | Hungary | | | |
| M1 | Methyl β-Cyclodextrin | Sigma-Aldrich, St. | C4555 | 98H0545 | 11.0 |
| | | Louis, MO | | | |
| M2 | Methyl β-Cyclodextrin | Sigma-Aldrich, St. | 332615 | STBH0439 | 10.8 |
| | | Louis, MO | | | |
| M3 | Methyl β-Cyclodextrin | Sigma-Aldrich, St. | 332615 | STBF5361V | 11.9 |
| | | Louis, MO | | | |
| M4 | Heptakis (2,6-di-O- | Sigma-Aldrich, St. | H0513 | SLBD0504V | 15.1 |
| | Methyl) β-Cyclodextrin | Louis, MO | | | |
| M5 | Heptakis (2,3,6-tri-O- | Fluka, Mexico City, | 51707 | 1421422 | 19.6 |
| | Methyl) β-Cyclodextrin | Mexico | | | |
| H1 | Hydroxy propyl β- | Sigma-Aldrich, St. | C0926 | 077k0680v | 4-10 |
| | Cyclodextrin | Louis, MO | | | |
| H2 | Hydroxy propyl β- | Carbosynth, San | OH053931 | OH053931501 | 5.4 |
| | Cyclodextrin | Diego, CA | | | |
| H3 | Hydroxy propyl β- | Carbosynth, San | OH053931 | OH053931801 | 4.9 |
| | Cyclodextrin | Diego, CA | | | |
| SB1 | Sulfo Butyl Ether β- | Carbosynth, San | OC15979 | OS159791402 | 7.1 |
| | Cyclodextrin | Diego, CA | | | |
| SB2 | Sulfo Butyl Ether β- | Carbosynth, San | OC15979 | OC159791801 | 6.6 |
| | Cyclodextrin | Diego, CA | | | |
| SB3 | Sulfo Butyl Ether β- | Carbosynth, San | OC15979 | OC159791601 | 6.7 |
| | Cyclodextrin | Diego, CA | | | |
| G0 | Gamma-Cyclodextrin | TCI America, | C0869 | 6ZNJH-0B | 0 |
| | | Portland, OR | | | |

### Example 2: Effects of Cyclodextrin Compounds of the Present Technology on Cellular LB Deposits

The in vitro assay described in FIG. 5A allows rapid evaluation of the capacity of a cyclodextrin to remove cellular LBs. Since all cyclodextrins have a hydrophobic pocket in the center, it was hypothesized that all cyclodextrins would be effective in promoting removal of lipofuscin bisretinoids from epithelial cells. To test this hypothesis, human RPE cells were pre-loaded with lipofuscin by incubating them with 5 μM A2E overnight. Cells were washed and left untreated or treated with 4 mM of a test cyclodextrin compound. After 48 hrs treatment, cells were lysed in 2% Triton/2% SDS-PBS buffer. A2E content in the cells was measured by fluorescence (excitation at 430 nm and emission at 600 nm).

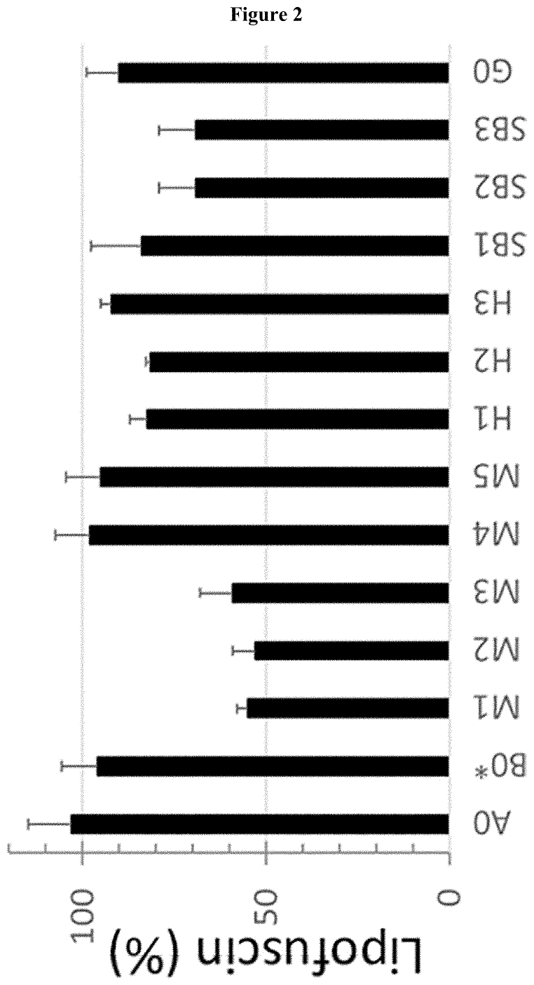

As shown in FIG. 2, not all cyclodextrins were effective in promoting LB removal. Whereas methyl β-Cyclodextrin, 2-Hydroxy Propyl β-Cyclodextrin, and Sulfo Butyl Ether β-Cyclodextrin promoted removal of lipofuscin bisretinoids from retinal cells, unsubstituted α-Cyclodextrin, β-Cyclodextrin and gamma-cyclodextrin did not extract A2E under these conditions. 2,6-di-O-methyl-β-Cyclodextrin (Heptakis 2,6-di-O-methyl) or 2,3,6-tri-O-methyl-β-Cyclodextrin (Heptakis 2,3,6-tri-O-methyl) were also not effective in promoting LB removal.

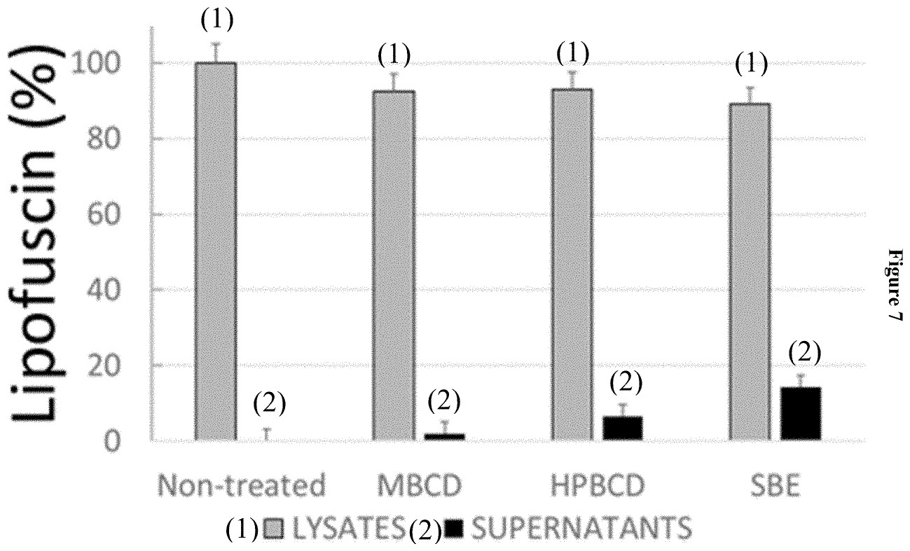

To confirm that the removal of LB deposits took place, cells were analyzed under a fluorescence microscope with a 10× magnification objective. Yellow autofluorescence characteristic of lipofuscin was dramatically reduced after treatment with 7.5 mM (β-Cyclodextrins, for 48 hrs (FIG. 6).

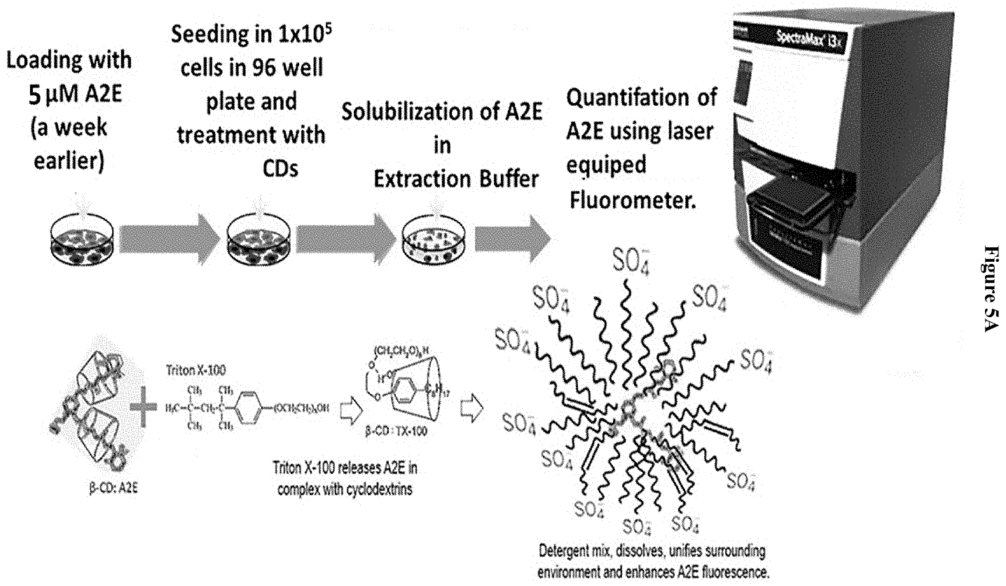

To further elucidate the LB removal process promoted by the tested s-cyclodextrins, A2E-loaded cells were seeded on optically pure glass bottom plates, treated for 48 hrs with 7.5 mM β-Cyclodextrins, and imaged using a high magnification objective (100×). FIGS. 4A-4B show fluorescence of A2E inside the cells (unextracted cells) monitored by fluorescence microscopy with a 100× magnification objective after staining with Hoechst dye for 1 hour to visualize nuclei. Top panel shows LB-rich lipofuscin accumulation in an untreated control (A2E-Untreated). As shown in FIGS. 4A-4B, the untreated cells showed both blue DNA fluorescence, and yellow punctate LB fluorescence, in the cytoplasm. This localization was consistent with previously reported localization inside the lysosomes. Bottom panel shows that LB was expelled outside the cells after the treatment with methyl β-Cyclodextrin. Indeed, extracellular release of granules of A2E appeared to be induced by the treatment.

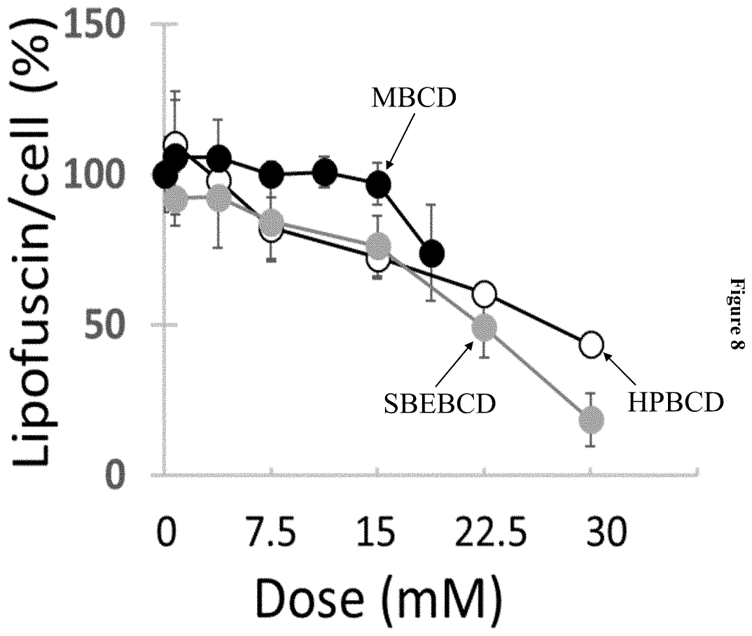

To confirm that the process of lipofuscin removal involves the extracellular release of lipofuscin, 1×10⁵ A2E-loaded and control cells per well were cultured in serum free media and after 4 hrs treatment with 12.5 mM Methyl β-Cyclodextrin (MBCD); Hydroxy propyl β-Cyclodextrin (HPBCD); or Sulfo Butyl Ether β-Cyclodextrin (SBE-BCD). Adherent cells were lysed in 1×A2E extraction buffer and supernatants were transferred to a fresh set of wells where concentrated A2E extraction buffer was added to obtain a 1× final concentration, thus allowing quantification of A2E secreted into the media. As shown in FIG. 7, all A2E fluorescence that was absent from cells was present in supernatants. HPLC analysis was then performed in lysates and supernatants to verify that A2E was not degraded during this process (data not shown).

These results demonstrate that the cyclodextrin compositions of the present technology are useful in methods for treating eye diseases associated with retinal cell lipofuscin accumulation in a subject in need thereof.

### Example 3: Dose-Dependent Removal of LB with β-Cyclodextrins

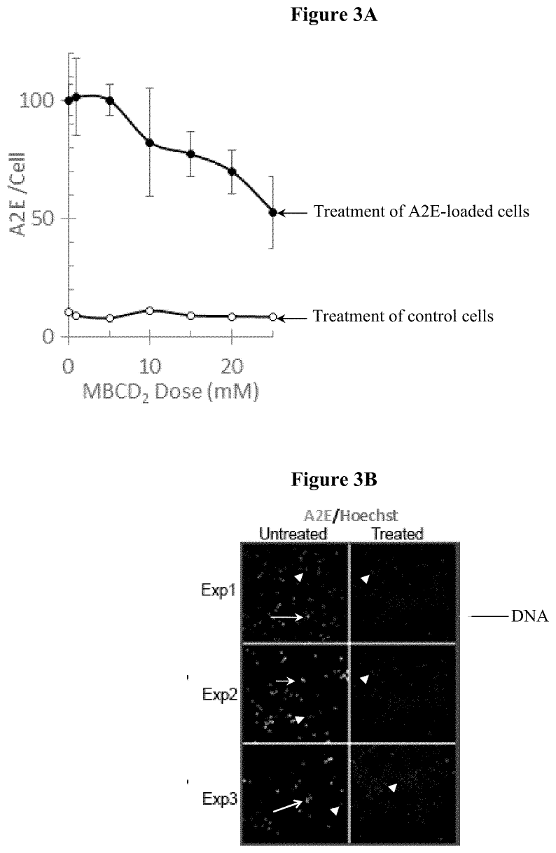

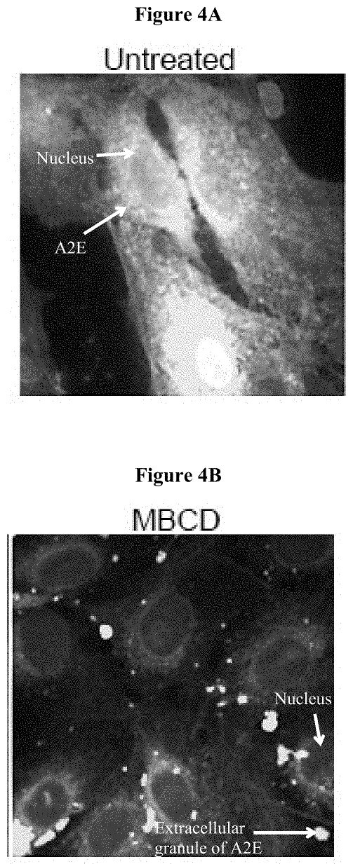

To determine the effective doses of methyl β-Cyclodextrin, epithelial cells loaded with A2E were incubated with increasing doses of methyl β-Cyclodextrin. After incubation for 24 hrs, methyl β-Cyclodextrin was removed from the cells and the amount of A2E still associated with the cells was determined. As shown in FIG. 3A, the effects of methyl β-Cyclodextrin on the removal of A2E was dose-dependent. The lowest tested dose showed about 75% removal, compared to untreated control. Doses as low as 10-20 μM methyl β-Cyclodextrin removed most of the detectable LB deposits from the epithelial cells.

To assess removal of LB deposits, cells were analyzed at 100× magnification under a fluorescence microscope after staining the nuclei with DAPI. Untreated control cells were also observed under the same conditions. As shown in FIG. 3B, the untreated cells exhibited both blue DNA fluorescence, as well as turquoise fluorescence due to LB deposits. In contrast, cells treated with methyl β-Cyclodextrin for 24 hours exhibited blue fluorescence, and were completely devoid of the turquoise fluorescence, thus demonstrating extraction and removal of LB.

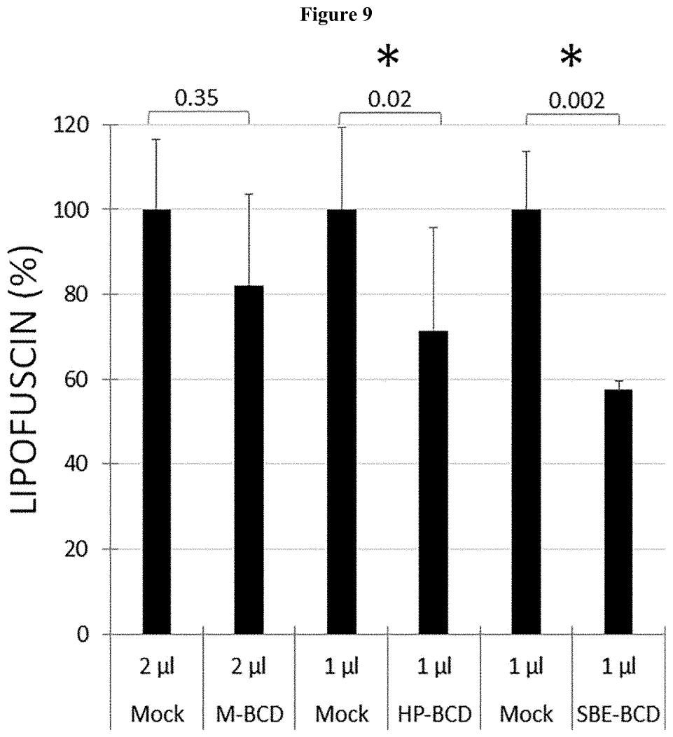

To compare the potency of β-cyclodextrins, epithelial cells loaded with A2E were incubated with increasing doses of the indicated s-cyclodextrins. After incubation for 48 hrs, the media with β-cyclodextrin supernatant were removed and the amount of A2E still associated with the cells was determined by the quantitative fluorometric assay described in FIG. 5A. As shown in FIG. 8, the effects of s-cyclodextrins on the removal of A2E was dose-dependent. Doses of β-cyclodextrins, in the millimolar range removed LB deposits from the epithelial cells.

These results demonstrate that the cyclodextrin compositions of the present technology are useful in methods for treating eye diseases associated with retinal cell lipofuscin accumulation in a subject in need thereof.

### Example 4: Removal of LB from Eyes Treated with Intravitreal Injections

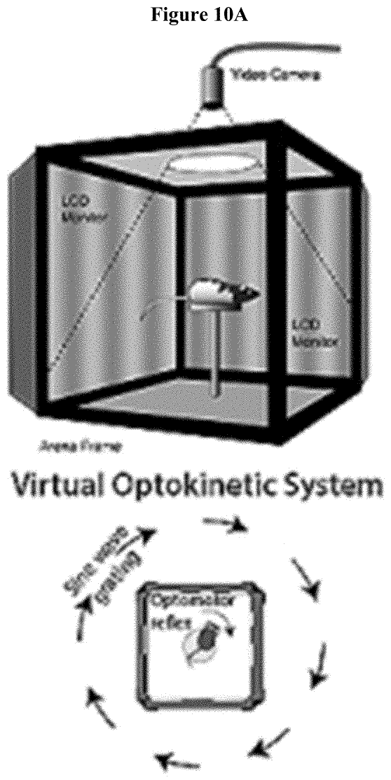

To further determine whether Methyl β-Cyclodextrin (MBCD); Hydroxy propyl β-Cyclodextrin (HPBCD); and/or Sulfo Butyl Ether β-Cyclodextrin (SBE-BCD) were able to extract LB from eyes in vivo the following experiment was performed. Right eyes (OD) of DKO mice, i.e. animals with exacerbated accumulation of LB, due to mutations in the ABCA4 and RDH8 genes (Maeda A, et al. (2008) Retinopathy in Mice Induced by Disrupted All-trans-retinal Clearance. J Biol Chem 283(39):26684-93) were treated with two intravitreal injections of 2 μl 100 mM MβCD; 1 μl 500 mM HPβCD; or 1 μl 500 mM SBE-βCD and left eyes (OS) were mock-treated with vehicle alone. A separate group of age matched control animals was also treated with vehicle alone to stablish the baseline autofluorescence in the eyes of these mice. The second identical injection was administered a week later. Eyes were harvested 4 days after the second injection and retinal pigment epithelium (RPE)-eyecups were flat mounted. Autofluorescence microscopy of the flat mounted RPE-eyecups was performed at 630× magnification and the image of the whole eyecup was constructed by stitching individual images, using ZEN Blue software (Zeiss). Mean fluorescence intensity of the retinas was quantified using Image J Software. FIG. 9 demonstrates that SBE-BCD and HP-BCD but not MBCD were able to significantly remove LB from retinas at the tested concentration. SBE-BCD was better than HPBCD. As shown in FIG. 9, 42.6, 28.5 and 18.2% of the total LB were extracted with two intravitreal injections of SBE-BCD, HP-BCD and MBCD, respectively.

These results demonstrate that the cyclodextrins of the present technology promote LB removal and are useful in methods for treating lipofuscin associated blinding disorders via intraocular injection, implantation of slow release devices or topical (eye drops).

### Example 5: In Vivo Effects of the Cyclodextrins of the Present Technology on Visual Acuity

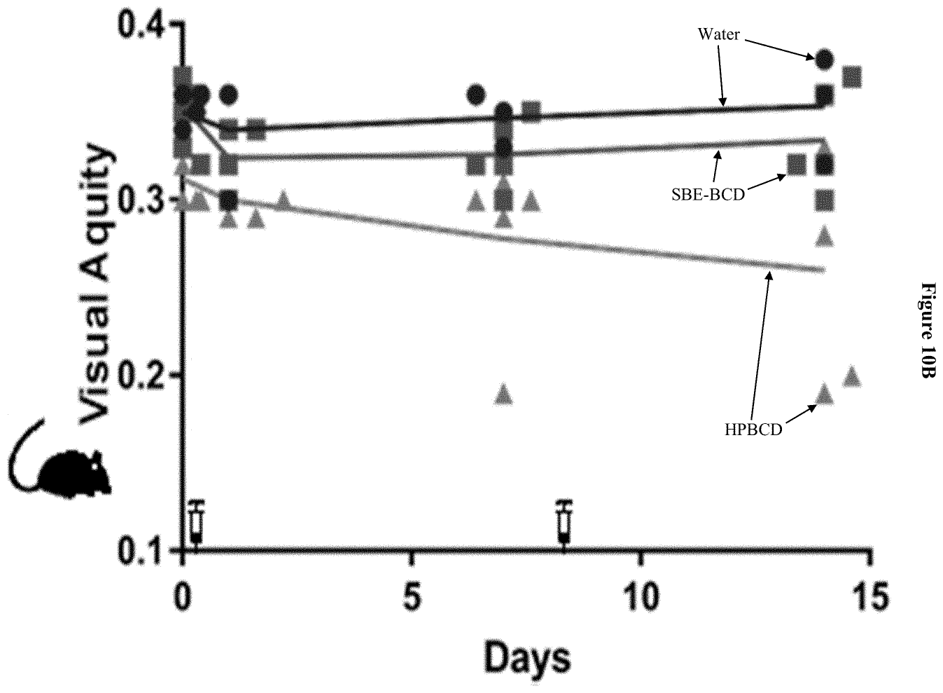

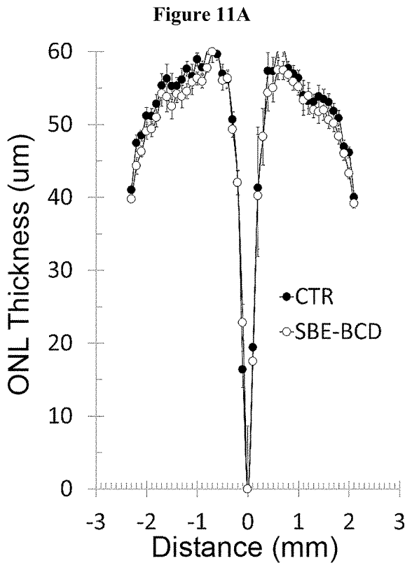

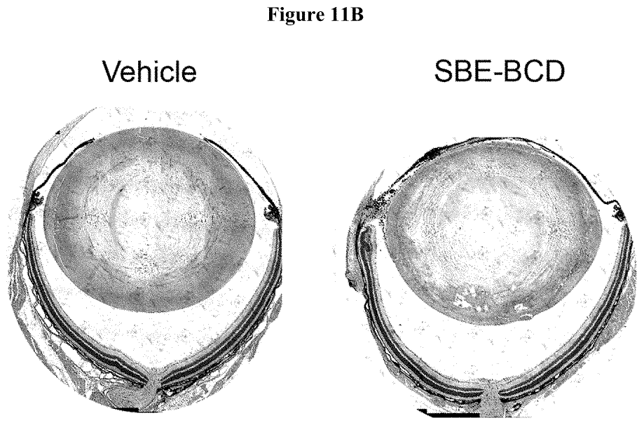

2 μl 100 mM M-βCD; 1 μl 500 mM HP-3CD; or 1 μl 500 mM SBE-βCD were injected twice, one-week apart, in both eyes and 2 μl H2O were injected intravitreally to age matched controls. To evaluate the visual toxicity of the treatment, the Spatial Frequency response (SF) was determined just before each injection and before sacrificing the mouse, i.e., four days after the second injection. SF is a cone-mediated visual response measured using an OptoMotry (OMT) chamber. Briefly, the mouse standing unrestrained on an elevated platform is surrounded by computer monitors that create a virtual cylinder with black bars on white background. The virtual rotation of the grating triggers vision-reflexive head movements; the highest spatial frequency (SF) that does not elicit tracking is taken as a measure of visual acuity. SF was not measurable in M-βCD treated mice. With HP-βCD there was observable variation with some animals showing significant impairment of visal response. In contrast, SBE-βCD was well tolerated showing indistinguishable SF from water treated and non-injected negative controls. See FIGS. 10A-10B. Treatment with Sulfo Butyl Ether β-Cyclodextrin (SBE-BCD) did not compromise the integrity of the photoreceptor layer (ONL), and the overall structure of the retinas of SBE-BCD-treated animals remained intact following treatment. See FIGS. 11A-11B.

These results demonstrate that the cyclodextrins of the present technology promote LB removal and are useful in methods for treating lipofuscin associated blinding disorders via intraocular injection, implantation of slow release devices or topical application (eye drops). Accordingly, the cyclodextrin compositions of the present technology are useful in methods for treating eye diseases associated with retinal cell lipofuscin accumulation in a subject in need thereof.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A method for preventing or treating an eye disease associated with retinal cell lipofuscin accumulation without impairing visual acuity in a subject in need thereof comprising administering to the subject an effective amount of sulfobutyl ether β-Cyclodextrin (SBE-βCD) or a pharmaceutically acceptable salt thereof.

2. The method of claim 1, wherein the eye disease associated with retinal cell lipofuscin accumulation is selected from the group consisting of Stargardt disease (STGD), retinitis pigmentosa (RP), Age-Related Macular Degeneration (AMD), Best disease (BD), and cone-rod dystrophy.

3. The method of claim 1, wherein the eye disease is genetic, non-genetic, or associated with aging.

4. A method for preventing or treating retinal cell lipofuscin accumulation without impairing visual acuity in a subject in need thereof comprising administering to the subject an effective amount of sulfobutyl ether β-Cyclodextrin (SBE-βCD) or a pharmaceutically acceptable salt thereof.

5. The method of claim 1, wherein administration of the effective amount of the SBE-βCD or pharmaceutically acceptable salt thereof prevents exacerbation of lipofuscin-associated retinal damage in the subject.

6. A method for reducing lipofuscin accumulation in retinal pigment epithelium cells comprising contacting the retinal pigment epithelium cells with an effective amount of sulfobutyl ether β-Cyclodextrin (SBE-βCD) or a pharmaceutically acceptable salt thereof.

7. The method of claim 1, wherein the SBE-βCD or pharmaceutically acceptable salt thereof is configured to localize to retinal pigment epithelium cells.

8. The method of claim 7, wherein the SBE-βCD or pharmaceutically acceptable salt thereof is configured to complex with lipofuscin bisretinoid lipids in the retinal pigment epithelium cells.

9. The method of claim 1, wherein administration of the effective amount of the SBE-βCD or pharmaceutically acceptable salt thereof blocks, mitigates, or reverses accumulation of lipofuscin in retinal pigment epithelium cells.

10. The method of claim 8, wherein the lipofuscin bisretinoid lipids are selected from the group consisting of N-retinylidene-N-retinylethanolamine (A2E), an A2E isomer, an oxidized derivative of A2E, and all-trans-retinal dimers.

11. The method of claim 1, wherein the SBE-βCD or pharmaceutically acceptable salt thereof is coupled to an agent that targets retinal pigment epithelium cells.

12. The method of claim 11, wherein the agent targets endosomes or lysosomes in the retinal pigment epithelium cells.

13. The method of claim 11, wherein the agent is mannose 6-phosphate.

14. The method of claim 1, wherein the SBE-βCD or pharmaceutically acceptable salt thereof is administered via topical, intravitreous, intraocular, subretinal, or subscleral administration.

15. The method of claim 14, wherein subscleral administration is achieved by implanting a slow-release subscleral implant in the subject.

16. The method of claim 1, wherein the SBE-βCD or pharmaceutically acceptable salt thereof is coupled to a fluorophore.
